(19) United States
(12) Patent Application Publication
(10) Pub. No.: US 2023/0149652 A1
(43) Pub. Date: May 18, 2023

(54) NASAL RESPIRATORY MASK

(71) Applicant: Flexicare (Group) Limited, Mid Glamorgan (UK)

(72) Inventors: Ghassem Poormand, London (GB); Shahrzad Poole, Irvine, CA (US)

(21) Appl. No.: 18/156,005

(22) Filed: Jan. 18, 2023

Related U.S. Application Data
(63) Continuation-in-part of application No. 17/505,332, filed on Oct. 19, 2021.
Application No. 17/505,332 is a continuation-in-part of application No. PCT/GB2021/051911, filed on Jul. 23, 2021.

(51) Int. Cl. A61M 16/06; A61M 16/08; A61M 16/20; A61M 16/00

(52) U.S. Cl. CPC A61M 16/0622; A61M 16/0875; A61M 16/208; A61M 16/0057; A61M 2210/0618

(57) ABSTRACT

A nasal respiratory mask for a high flow oxygen therapy apparatus, comprising: a mask frame; and a mask cushion on the mask frame for contacting and substantially sealing against a face of a patient, the mask frame and mask cushion defining a nasal breathing cavity, wherein the mask frame comprises: a hose attachment portion for attaching a hose for delivering a supply of oxygen enriched air to the patient; and a mask aperture for restricting the flow of gas from the nasal breathing cavity directly to ambient, wherein the mask aperture maintains a positive end-expiratory pressure (PEEP) in the nasal breathing cavity of between 0.2 kPa and 1 kPa during the administering of high flow oxygen therapy to the patient.

### CROSS-REFERENCE TO RELATED APPLICATIONS

This application is a Continuation-in-Part of U.S. Non-Provisional patent application Ser. No. 17/505,332, filed Oct. 19, 2021, which is a Continuation-in-Part of International Patent Application No. PCT/GB2021/051911, filed Jul. 23, 2021. Both applications are incorporated herein by reference in their entirety.

### FIELD OF THE INVENTION

The present invention relates to a nasal respiratory mask, a nasal respiratory mask for a high flow oxygen therapy apparatus, a nasal respiratory mask system, and a high flow oxygen therapy apparatus.

### BACKGROUND OF THE INVENTION

High Flow Oxygen Therapy (HFOT) delivers an air/oxygen gas mix to the patient at flow rates that exceed the patient's peak inspiratory flow rates at various minute volumes. The air flow is heated, humidified, and oxygen enriched. The respiratory support offered by HFOT is non-invasive.

HFOT is typically delivered to the patient by nasal cannula, which offers many advantages over traditional face masks in that the nasal cannulas allow a patient to eat and are tolerated by patients for longer periods. However, extended wear of a nasal cannula can still lead to patient discomfort, for example, pressure sores on the nasal septum. Nasal respiratory masks offer an alternative to nasal cannulas.

### SUMMARY OF THE INVENTION

A first aspect of the invention provides a method of administering high flow oxygen therapy to a patient using a high flow oxygen therapy apparatus, the apparatus comprising: a nasal respiratory mask comprising a mask frame; a mask cushion on the mask frame, the mask frame and mask cushion defining a nasal breathing cavity; a hose attachment portion; and a mask aperture having an adjustable flow restrictor for restricting the flow of gas from the nasal breathing cavity directly to ambient; and an oxygen enriched air supply having a variable flow rate output for supplying heated, humidified oxygen enriched air to the patient, the method comprising: providing the nasal respiratory mask to the patient's face so that the nasal breathing cavity surrounds the patient's nose and not the mouth, and the mask cushion contacts and substantially seals against the face of the patient; connecting a hose between the hose attachment portion of the mask and the oxygen enriched air supply; adjusting the variable flow rate output of the oxygen enriched air supply to provide a flow rate of the heated, humidified oxygen enriched air to the mask which just exceeds the patient's peak nasal inspiratory flow rate; adjusting the adjustable flow restrictor of the mask to maintain a positive end-expiratory pressure (PEEP) in the nasal breathing cavity of between 0.2 kPa and 1 kPa during the administering of high flow oxygen therapy to the patient.

A further aspect of the invention provides a method of administering high flow oxygen therapy to a patient using a high flow oxygen therapy system, the system comprising: a plurality of nasal respiratory masks, each mask comprising: a mask frame; a mask cushion on the mask frame, the mask frame and mask cushion defining a nasal breathing cavity; a hose attachment portion; and a mask aperture for restricting the flow of gas from the nasal breathing cavity directly to ambient, each of the plurality of masks having a differently sized mask aperture for restricting the flow of gas from the nasal breathing cavity directly to ambient by a different amount; and an oxygen enriched air supply having a variable flow rate output for supplying heated, humidified oxygen enriched air to the patient, the method comprising: selecting one of the plurality of nasal respiratory masks; providing the selected nasal respiratory mask to the patient's face so that the nasal breathing cavity surrounds the patient's nose and not the mouth, and the mask cushion contacts and substantially seals against the face of the patient; connecting a hose between the hose attachment portion of the mask and the oxygen enriched air supply; adjusting the variable flow rate output of the oxygen enriched air supply to provide a flow rate of the heated, humidified oxygen enriched air to the mask which just exceeds the patient's peak nasal inspiratory flow rate; wherein the selected nasal respiratory mask is selected to maintain a positive end-expiratory pressure (PEEP) in the nasal breathing cavity of between 0.2 kPa and 1 kPa during the administering of high flow oxygen therapy to the patient.

A further aspect of the invention provides a method of administering high flow oxygen therapy to a patient using a high flow oxygen therapy system, the system comprising: a plurality of nasal respiratory masks, each mask comprising: a mask frame; a mask cushion on the mask frame, the mask frame and mask cushion defining a nasal breathing cavity; a hose attachment portion; and a mask aperture, wherein the mask aperture has a passive one-way valve configured to move from a closed position in which air is restricted from flowing through the one-way valve, to an open position in which air can flow from the nasal breathing cavity through the one-way valve directly to ambient, each of the plurality of masks having a different one-way valve with a different pre-set valve opening pressure of less than 1 kPa; and an oxygen enriched air supply having a variable flow rate output for supplying heated, humidified oxygen enriched air to the patient, the method comprising: selecting one of the plurality of nasal respiratory masks; providing the selected nasal respiratory mask to the patient's face so that the nasal breathing cavity surrounds the patient's nose and not the mouth, and the mask cushion contacts and substantially seals against the face of the patient; connecting a hose between the hose attachment portion of the mask and the oxygen enriched air supply; adjusting the variable flow rate output of the oxygen enriched air supply to provide a flow rate of the heated, humidified oxygen enriched air to the mask; wherein the selected nasal respiratory mask is selected to maintain a positive end-expiratory pressure (PEEP) in the nasal breathing cavity of between 0.2 kPa and 1 kPa during the administering of high flow oxygen therapy to the patient.

A further aspect of the invention provides a method of administering high flow oxygen therapy to a patient using a high flow oxygen therapy system, the system comprising: a nasal respiratory mask comprising: a mask frame; a mask cushion on the mask frame, the mask frame and mask cushion defining a nasal breathing cavity; a hose attachment portion; and a mask aperture, wherein the mask aperture has a passive one-way valve configured to move from a closed position in which air is restricted from flowing through the one-way valve, to an open position in which air can flow from the nasal breathing cavity through the one-way valve directly to ambient, wherein the one-way valve has a variable valve opening pressure; and an oxygen enriched air supply having a variable flow rate output for supplying heated, humidified oxygen enriched air to the patient, the method comprising: selecting one of the plurality of nasal respiratory masks; providing the selected nasal respiratory mask to the patient's face so that the nasal breathing cavity surrounds the patient's nose and not the mouth, and the mask cushion contacts and substantially seals against the face of the patient; connecting a hose between the hose attachment portion of the mask and the oxygen enriched air supply; adjusting the variable flow rate output of the oxygen enriched air supply to provide a flow rate of the heated, humidified oxygen enriched air to the mask; adjusting the valve opening pressure of the mask to maintain a positive end-expiratory pressure (PEEP) in the nasal breathing cavity of between 0.2 kPa and 1 kPa during the administering of high flow oxygen therapy to the patient.

A further aspect of the invention provides a nasal respiratory mask for a high flow oxygen therapy apparatus, comprising: a mask frame; a mask cushion on the mask frame for contacting and substantially sealing against a face of a patient, the mask frame and mask cushion defining a nasal breathing cavity; the mask frame having: a hose attachment portion for attaching a hose for delivering a supply of oxygen enriched air to the patient; and a mask aperture having an adjustable flow restrictor for restricting the flow of gas from the nasal breathing cavity directly to ambient, wherein the adjustable flow restrictor is adjustable to maintain a positive end-expiratory pressure (PEEP) in the nasal breathing cavity of between 0.2 kPa and 1 kPa during the administering of high flow oxygen therapy to the patient.

A further aspect of the invention provides a nasal respiratory mask for a high flow oxygen therapy apparatus, comprising: a mask frame; a mask cushion on the mask frame for contacting and substantially sealing against a face of a patient, the mask frame and mask cushion defining a nasal breathing cavity; the mask frame having: a hose attachment portion for attaching a hose for delivering a supply of oxygen enriched air to the patient; and a mask aperture having a passive one-way valve configured to move from a closed position in which air is restricted from flowing through the one-way valve, to an open position in which air can flow from the nasal breathing cavity through the one-way valve directly to ambient outside the mask, wherein the one-way valve has a valve opening pressure for maintaining a positive end-expiratory pressure (PEEP) in the nasal breathing cavity of between 0.2 kPa and 1 kPa during the administering of high flow oxygen therapy to the patient.

A further aspect of the invention provides a nasal respiratory mask for a high flow oxygen therapy apparatus, comprising: a mask frame; a mask cushion on the mask frame for contacting and substantially sealing against a face of a patient, the mask frame and mask cushion defining a nasal breathing cavity; the mask frame having: a hose attachment portion for attaching a hose for delivering a supply of oxygen enriched air to the patient; and a mask aperture for restricting the flow of gas from the nasal breathing cavity directly to ambient, wherein the mask aperture is dimensioned for maintaining a positive end-expiratory pressure (PEEP) in the nasal breathing cavity of between 0.2 kPa and 1 kPa during the administering of high flow oxygen therapy to the patient.

A nasal respiratory mask is a mask that covers the nasal passages of a patient but not the mouth passage, in contrast to a face mask. The nasal respiratory mask may be arranged to extend from a position above the nasal passages to a position above the mouth passage. The nasal respiratory mask may be arranged to extend from the bridge of a patient's nose to their upper lip, or extend between a position below the bridge of a patient's nose to a position above the patient's upper lip.

The adjustable flow restrictor may be adjusted to maintain a positive end-expiratory pressure (PEEP) in the nasal breathing cavity of approximately 0.5 kPa during the administering of high flow oxygen therapy to the patient, preferably 0.3 to 0.7 kPa, preferably 0.4 to 0.6 kPa. The flow restrictor may be manually adjusted.

The mask aperture may further comprise either a vent or a passive one-way valve. The vent or valve may be located adjacent the flow restrictor and fluidically coupled in the same flow passage as the flow restrictor, preferably immediately upstream of the flow restrictor in the flow direction of exhaled gas from the nasal breathing cavity towards ambient.

The vent defines an opening that remains open during use. In particular, the vent remains open during both inhalation and exhalation of the patient. The vent may be sized to restrict the flow of gas from the nasal breathing cavity to ambient, so as to maintain a positive end-expiratory pressure (PEEP) in the nasal breathing cavity of at least 0.2 kPa during the administering of high flow oxygen therapy to the patient, preferably approximately 0.5 kPa, preferably 0.3 to 0.7 kPa, preferably 0.4 to 0.6 kPa.

The passive one-way valve may have a valve member configured to move from a closed position in which air is restricted from flowing through the one-way valve, to an open position in which air can flow from the nasal breathing cavity through the one-way valve towards the flow restrictor and to outside the mask. The passive one-way valve may be fully passive and operable to open and close only in direct dependence on the pressure of air in the mask aperture flow passage between the nasal breathing cavity and ambient.

The one-way valve may have a movable valve member that is unbiased and free floating. Alternatively, the valve member may be biased to provide a valve opening pressure of less than 1 kPa and/or a valve opening pressure which is greater than 0.2 kPa. The valve opening pressure may be pre-set or may be variable, e.g. by manual adjustment. The valve opening pressure may not be adjustable by a pressure feedback mechanism.

The valve opening pressure may be less than 0.8 kPa.

The vent may have a flow passage cross-sectional area that is larger than the flow passage cross sectional area of the flow restrictor (where provided) when in the fully open position. This ensures that the vent does not impinge the flow when the flow restrictor is fully open.

A flow rate through the one-way valve in the open position and/or a flow rate through the vent and/or through the flow restrictor may be configured to be at least 2 litres per minute, preferably at least 5 litres per minute.

The one-way valve may be a flapper valve or a lift-check valve.

The mask frame may have a generally domed shape.

The mask cushion may comprise a thermoplastic elastomer and/or silicone.

The mask cushion and at least a perimeter of the mask frame may be integrally formed of the same material.

At least a portion of the mask frame may comprise a substantially less flexible material than the material of the mask cushion.

The mask cushion may be inflatable and deflatable.

The hose attachment portion may be substantially centrally located on a vertical centre line of the mask frame.

The hose attachment portion may be located towards a lower end of the mask frame. The hose attachment portion may be located towards a lower end of the mask frame and arranged so as to be adjacent a middle of a user's mouth when worn.

The nasal respiratory mask may comprise two of the mask apertures spaced substantially symmetrically about a vertical centre line of the mask frame.

The two mask apertures may be located towards a lower end of the mask frame so as to be adjacent either side of a user's mouth when worn.

The mask frame may be at least partially formed from a water permeable material.

At least 50% of the mask frame may be formed from the water permeable material.

The water permeable material may be permeable to liquid water and/or water vapour.

The hose attachment portion may comprise a swivel connector configured to provide relative rotation between the mask frame and the hose.

The nasal respiratory mask may comprise a pair of opposing straps and/or harness extending from the mask frame.

The nasal respiratory mask may comprise a carbon dioxide monitoring line connector on the mask frame for attaching a carbon dioxide monitoring line and/or a carbon dioxide sensor.

The nasal respiratory mask may comprise a carbon dioxide sensor on the mask frame.

The nasal respiratory mask may comprise a carbon dioxide monitoring line attached to the carbon dioxide monitoring line connector and a carbon dioxide sensor attached to the carbon dioxide monitoring line.

The carbon dioxide monitoring line may comprise a water permeable material.

The nasal respiratory mask may further comprise a filter membrane arranged to cover the mask aperture.

The filter membrane may be arranged to cover at least half of the mask frame.

The filter membrane may be arranged to cover a patient's mouth

A further aspect of the invention provides a nasal respiratory mask system comprising the nasal respiratory mask of the invention and a hose for attaching to the hose attachment portion of the nasal respiratory mask for delivering a supply of oxygen enriched air to the user.

The hose may comprise a water permeable material.

The water permeable material may be permeable to liquid water and/or water vapour.

The hose may be malleable and/or comprise a malleable member, such that the hose is configured to be deformable and retain a given shape when the hose is manipulated.

A further aspect of the invention provides a high flow oxygen therapy apparatus comprising: the nasal respiratory mask system; and an oxygen enriched air supply coupled via the hose to the respiratory mask and configured to supply oxygen enriched air to a user.

The oxygen enriched air supply may be configured to deliver a flow rate of at least 5 litres per minute to the user, and preferably a flow rate of between 30 and 60 litres per minute.

The oxygen enriched air supply may be configured to deliver a flow rate of less than 70 litres per minute to the user.

### BRIEF DESCRIPTION OF THE DRAWINGS

Embodiments of the invention will now be described with reference to the accompanying drawings, in which:

FIG. 1 shows a perspective view of a nasal respiratory mask system according to a first example;

FIG. 2 shows a front view of the nasal respiratory mask system;

FIG. 3 shows a rear view of the nasal respiratory mask system attached to a head band;

FIG. 4 shows a bottom view of the nasal respiratory mask system;

FIG. 5 shows a side view of the nasal respiratory mask system with a carbon dioxide sensor connected to the nasal mask via a carbon dioxide monitoring line;

FIG. 6 shows the nasal respiratory mask system comprising a malleable member;

FIG. 7 shows a schematic of a high flow oxygen therapy apparatus comprising the nasal respiratory mask system;

FIGS. 8A and 8B show a one-way umbrella valve;

FIGS. 9A and 9B show a flapper valve;

FIGS. 10A and 10B show a lift-check valve;

FIGS. 11A and 11B show a spring-loaded inline one-way valve;

FIGS. 12A and 12B show the spring-loaded inline one-way valve comprising an adjustable flow restrictor;

FIG. 13 shows a nasal mask having unbiased one-way valves;

FIGS. 14A and 14B show an unbiased flapper valve;

FIGS. 15A and 15B show an unbiased inline one-way valve;

FIGS. 16A to 16G show a second example of an adjustable flow restrictor;

FIG. 17 shows a front view of a nasal respiratory mask system according to a second example;

FIG. 18 shows a perspective view of the nasal respiratory mask system;

FIG. 19 shows a perspective view of a neo-natal nasal respiratory mask system according to a third example;

FIG. 20 shows a front view of a neo-natal nasal respiratory mask system according to a fourth example;

FIG. 21 shows a front view of a nasal respiratory mask system according to a fifth example;

FIG. 22 shows a front view of a neo-natal nasal respiratory mask system according to a sixth example;

FIG. 23A shows a filter membrane covering the opening/aperture in one of the valves;

FIGS. 23B and 23C show a filter membrane arranged to cover the mask frame;

FIG. 23D shows a filter membrane arranged to cover the mask frame and a patient's mouth;

FIG. 24 shows a perspective view of a nasal respiratory mask system without a valve.

### DETAILED DESCRIPTION OF EMBODIMENT(S)

A high flow oxygen therapy (HFOT) apparatus delivers heated, humidified, and oxygen enriched air at flow rates many times higher than those used with standard face masks and nasal cannulas. For instance, the flow rates to a standard mask or cannula may be approximately 5 Standard litres per minute (LPM). In contrast, a HFOT apparatus may deliver flow rates of between 30 LPM and 70 LPM, so that a greater area of the patent's lungs is recruited for gas exchange, further improving blood oxygenation. The flow rate of the supply is controllable and may be set manually. The setting of the HFOT supply flow rate is determined based on the patient's inspiratory flow rate, for example the HFOT supply flow rate may be set to substantially match or just exceed the patient's peak inspiratory flow rates and so flow rates outside this range (higher and lower) may be applicable in some situations, for example pre-natal patients may receive a flow rate of 5 LPM or greater, and flow rates in some applications may meet and exceed 100 LPM. It will be understood that higher flow rates (e.g. those exceeding 70 LPM, or exceeding 100 LPM) may be arranged for use in short-term and temporary applications, such as pre-operative oxygenation or when a patient is in respiratory distress.

The patient's inspiratory flow rate is taken to be the inspiratory flow rate during an inhalation of the patient. The inspiratory flow rate may refer to the peak inspiratory flow rate during an inhalation of the patient.

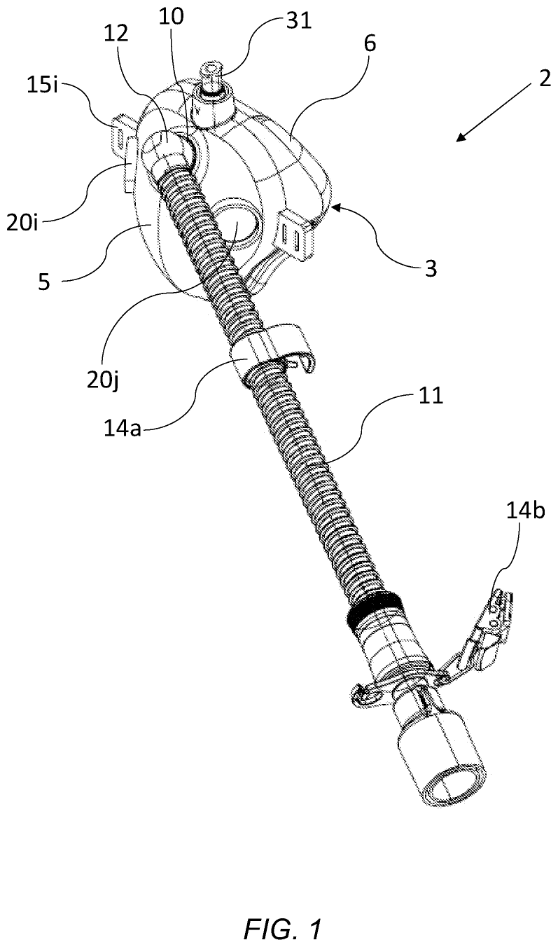

FIG. 1 shows an example of a nasal respiratory mask system 2 that may form part of a high flow oxygen therapy apparatus 1.

The nasal respiratory mask system 2 includes a nasal respiratory mask 3. The nasal respiratory mask 3 includes a mask frame 5, and a mask cushion 6 on the mask frame 5. The mask frame 5 and mask cushion 6 define a nasal breathing cavity between an internal side of the mask 3 and the patient. The mask 3 is intended to extend over the face of the patient, thereby covering the nasal passages. The mask 3 is arranged so as to cover the nasal passages but not the mouth passage. The nasal respiratory mask 3 may be arranged to extend from a position above the nasal passages to a position above the mouth passage. The nasal respiratory mask 3 may be arranged to extend from the bridge of a patient's nose to their upper lip, or extend between a position below the bridge of a patient's nose to a position above the patient's upper lip.

The mask frame 5 may comprise a material that is less flexible than the material forming the mask cushion 6. The mask frame 5 may be formed, entirely or in part, of a substantially rigid material in comparison to the structure and/or material of the mask cushion 6. A substantially rigid material is self-supporting and able to provide the necessary structure that forms and maintains the size of the nasal breathing cavity when the mask frame 5 is in use. The mask frame 5 may form a generally triangular dome shape. In other words, the mask frame 5 may have a shape that is triangular dome shaped in overall appearance but still have deviations from a perfectly triangular dome shape. However, it will be understood that the mask frame 5 may have any suitable shape, such as a circular or oval dome shape. The mask frame 5 may be formed from any suitable material, such as silicone or a thermoplastic elastomer (TPE).

The mask cushion 6 is arranged to contact and substantially seal against the face of a user. In this context, substantially seal is intended to refer to the mask cushion 6 as forming a sufficient seal to prevent excessive leaks, for example leaks that may detrimentally lower the flow rate and/or direct flow towards a patent's eye, thereby causing discomfort or poor performance. The mask cushion 6 may be inflatable and deflatable, thereby assisting in conforming to a patient's facial anatomy, for example the mask cushion may comprise an air valve that is attachable to an air pump (not shown).

The mask cushion 6 is arranged to prevent excess contact pressure being applied to the patient. The mask cushion 6 may be formed from any suitable material, for example silicone, thermoplastic elastomer gel or foam. Preferably the mask cushion 6 is formed of silicone. The mask cushion 6 may be attached to, or integral with, the mask frame 5.

In some examples, the mask frame 5 and mask cushion 6 may be integrally formed from the same material, such that the mask frame 5 and mask cushion 6 form a single part. In alternative examples, the mask frame 5 and mask cushion 6 may be separately formed.

The mask frame 5 and mask cushion 6 may be formed of the same material, or the mask frame 5 and mask cushion 6 may be formed of different materials. In one example, the mask frame 5 is formed of a thermoplastic elastomer and the mask cushion 6 is formed of silicone.

In other examples, the mask cushion 6 and at least a perimeter of the mask frame 5 may be integrally formed of the same material. In one example, the mask frame 5 may be formed of two materials (e.g. silicone and a thermoplastic elastomer), with the silicone being formed around the perimeter of the mask frame 5 that contacts the mask cushion 6. The mask cushion 6 may be formed of silicone, such that the silicone of the mask frame 5 extends integrally to form the mask cushion 6.

The mask frame 5 may be at least partially formed from a water permeable material. For example, the material may be permeable to liquid water and/or water vapour. The mask frame 5 may be entirely formed from the permeable material or partially formed from the permeable material. For example, at least 30%, at least 50%, or at least 70% of the mask frame 5 may be formed from the permeable material. A portion of the mask frame 5 may be formed from a permeable material whilst the remainder of the mask frame 5 is formed of a material that is relatively non-permeable (i.e. less permeable than the permeable material). In one example, the perimeter of the mask frame 5 is formed of a non-permeable material whilst the remaining material of the mask frame 5 is formed of the permeable material.

The permeable material is a material configured to allow water (e.g. liquid water and/or water vapour) to flow therethrough. The permeable material may be configured to reduce, restrict or prevent flow of gases therethrough. A mask frame 5 formed at least partially of a permeable material reduces the build-up of water in the nasal respiratory mask 3. Reducing, restricting or preventing the flow of gases through the material prevents a loss of air pressure in the nasal respiratory mask system 2.

The water permeable material of the mask frame 5 may be formed from an amphiphilic material. The water permeable material may be a hydrophobic and hydrophilic poly(ethylene oxide) based block co-polymer. Alternative water permeable materials include: water permeable polytetrafluoroethylene (PTFE); Nafion®; Sympatex®; Arnitel®; Diaplex®; water permeable Hytrel®; and Goretex®, although it will be appreciated that any suitable permeable material may be used.

The nasal respiratory mask 3 includes a hose attachment portion 10. The hose attachment portion may be attached to a hose 11. The hose 11 may be coupled to a supply of oxygen enriched air 60. The hose 11 may deliver a supply of oxygen enriched air to the patient from a source of oxygen enriched air 60.

The hose attachment portion 10 may comprise a connector 12 that attaches to the hose 11. The connector 12 may be a swivel connector 12 that allows the hose 11 to rotate relative to the mask frame 5. This allows a distal end of the hose 11, relative to the connector 12, to be rotated into a convenient position relative to the patient when in use. The swivel connector 12 may provide full 360 degree (or more) rotation of the hose 11 relative to the mask 3, or the relative rotation of the hose 11 relative to the mask 3 may be restricted to a set angular range. Alternatively, the connector 12 may be fixed in position relative to the mask 3.

As shown in FIGS. 1 to 4, the connector 12 may be an elbow connector 12 (in addition or alternatively to being a swivel connector). The elbow connector 12 redirects the air flow through the connector 12 such that the gas flow through the hose attachment portion 10 is angled with respect to the gas flow through the hose 11 adjacent to the elbow connector 12. The angle of the elbow connector 8 may be between 25 degrees and 90 degrees. In the example shown, the elbow connector 12 has an angle of 90 degrees. The elbow connector 12 provides a sharp right angle where two respective perpendicular sections of the elbow connector 12 meet, although in alternative examples the elbow connector 12 may be swept and/or curved such that the angular change is more gradual and there is no sudden angle change.

The connector 12 may be configured to vary its angle. For example, the elbow 12 connector may comprise a hinge mechanism. The hinge mechanism may be configured to allow the connector 12 to move from a first position, configured to redirect gas flow at an angle of 30 degrees, to a second position, configured to redirect gas flow at an angle of 90 degrees. The hinge mechanism may be fixable at a plurality of angular positions.

In some examples, the connector 12 may not include an elbow. In this case, the hose 11 may be coupled at one end to a straight connector pivotally or fixedly connected to the hose attachment portion 10. Due to the flexibility of the hose 11, the hose 11 may form an elbow or at least provide some flexibility that can increase patient comfort and convenience whilst wearing the nasal cannula.

The connection arrangement between the hose attachment portion 10 and the connector 12 may be non air-tight, such that some air/gas is able to escape or enter through the connection. The air/gas leakage through the connection arrangement may be maintained below a target value. The air/gas leakage through the connection may be below 10% at flows of up to 50 Standard litres per minute. In alternative examples, the air/gas leakage may be below 5% at 50 Standard litres per minute.

The connection arrangement may be a snap-fit connection, which allows the parts 10, 12 to be interlocked by pushing the parts together. A snap-fit connection allows the parts 10, 12 to be assembled quickly. The snap-fit connection may be a one-way snap-fit connection that does not permit ready disassembly.

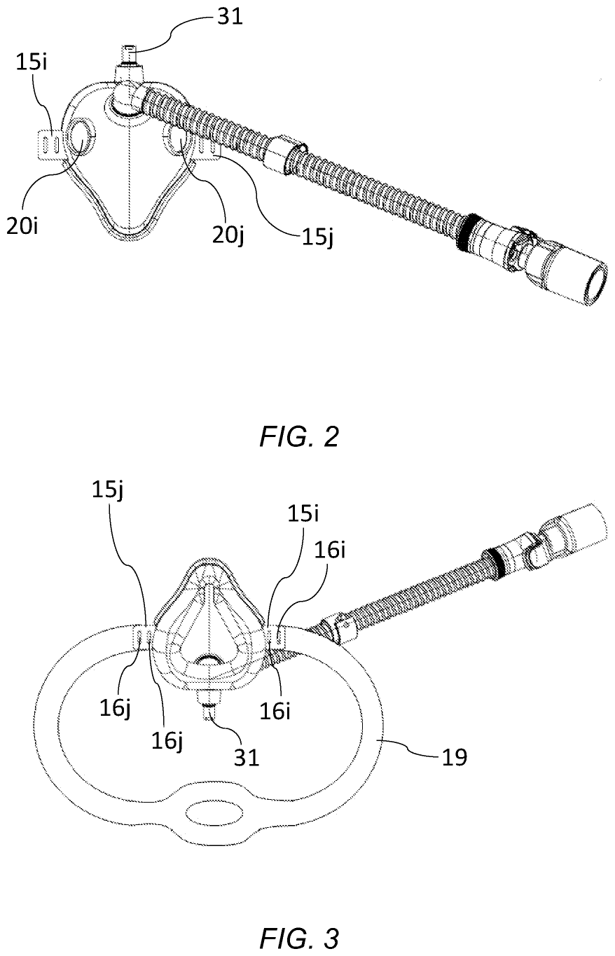

As shown best in FIG. 2, the hose attachment portion 10 may be substantially centrally located on a vertical centre line of the mask frame 5. The hose attachment portion 10 may therefore be positioned equidistant from the distal sides of the mask frame 5 and/or mask cushion 6 for locating the hose attachment portion 10 centrally on a patient's head. The hose attachment portion 10 may be located towards a lower end of the mask frame 5, as shown in the example of FIGS. 1 to 4. This may allow the hose attachment portion 10, through which the supply of oxygen enriched air enters the nasal breathing cavity, to be adjacent the middle of a user's mouth when worn.

The hose 11 may be flexible or rigid. The hose 11 may be sufficiently resilient to retain a substantially constant cross section of air flow, yet able to bend so that the hose 11 can be comfortably positioned and manoeuvred relative to a patient's face. The hose 11 may be a corrugated tube, e.g. with a corrugated outer surface. The inner surface of the hose 11 may also be corrugated, although in alternative examples the inner and/or outer surface may be smooth.

The hose 11 may be at least partially, and in some cases entirely, formed from a water permeable material, and function similarly to the permeable material of the mask frame 5 described above in that it allows liquid water and/or water vapour therethrough. The permeable material may be configured to restrict or prevent flow of gases therethrough. A hose 11 formed, at least partially, of a permeable material reduces the build-up of water and/or water vapour inside the hose 11. Reducing, restricting or preventing the flow of gases through the material prevents a loss of air pressure in the nasal respiratory mask system 2. Similarly, the connector 12 may be at least partially, and in some cases entirely, formed from the water permeable material.

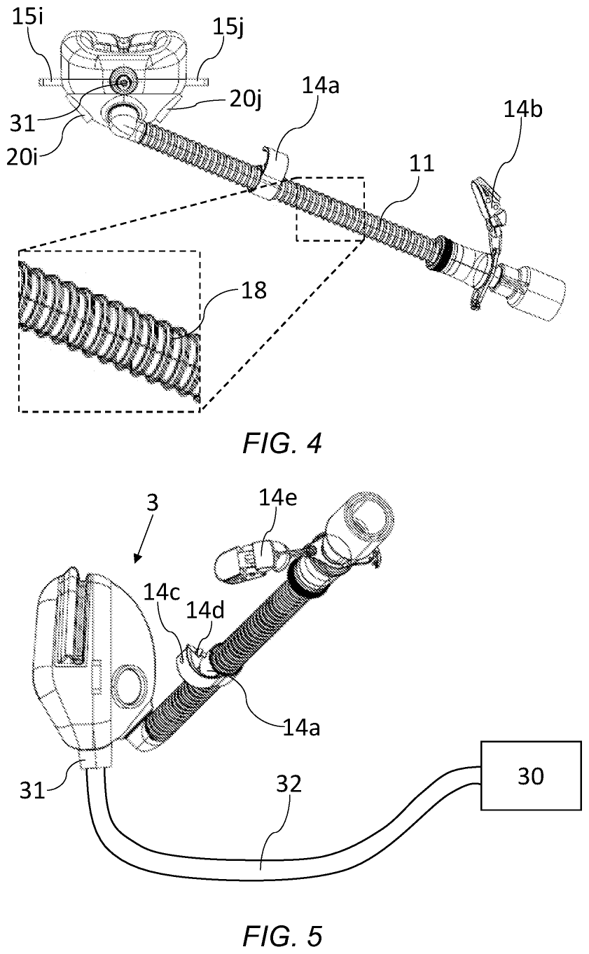

The nasal respiratory mask system 2 may include or be connected to a heating arrangement for heating up the oxygen enriched air prior to inhalation by the patient. For example, the hose 11 may comprise a heating wire breathing circuit that heats the air flowing through the hose 11. The heating wire breathing circuit may be provided in the form of a wire heating element 18 (See FIG. 4). The wire heating element 18 may extend around the hose 11, e.g. spiral around the hose 11. The wire heating element 18 may be positioned in any suitable position for heating up the air passing through the hose 11, for example the wire heating element 18 may be embedded within the wall of the hose 11, or wrapped around an outer surface of the hose 11, or within the hose 11 adjacent an inner surface of the hose 11.

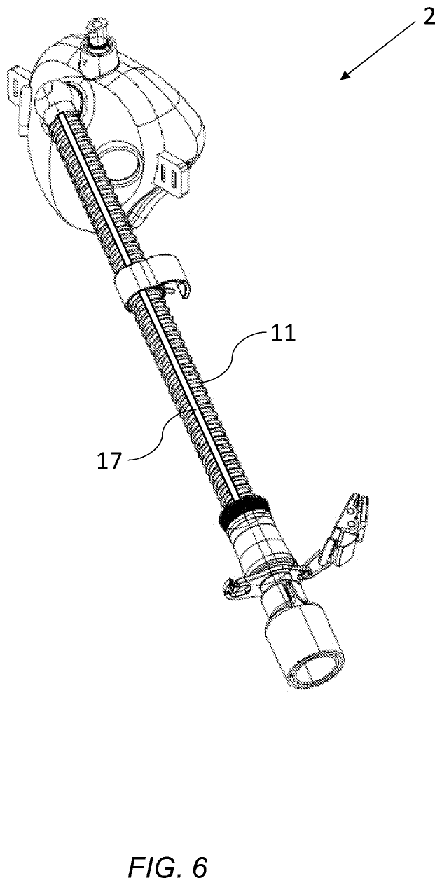

Alternatively, or in addition, the nasal respiratory mask system 2 may be connected to a heating unit as described in relation to FIG. 6.

The nasal respiratory mask 3 may comprise one or more clips 14a, 14b as shown, for example, in FIG. 4. A first clip 14a may be located between the ends of the hose 11. A second clip 14b may be located on a distal end of the hose 11, relative to the hose attachment portion 10. The one or more clips 14a, 14b may be configured for securing the hose 11 to an object. When fastened to an object, either directly or indirectly, the clips 14a, 14b may help to reduce strain on the hose 11 and help to position the hose 11 in use.

In some examples, clips 14a, 14b may include one or more holes 14c through which a fastening pin 14d extends (See clip 14a in FIG. 5) so as to fasten the clip 14a. The clip 14a may comprise a plurality of holes 14c so that the size and tightness of the clip 14a can be adjusted. Alternatively, the clip 14a, 14b may include a crocodile clip end 14e, or similar, that latches onto an object.

In some examples, either or both of the clips 14a, 14b may be garment clips for attaching to an item of clothing of the patient, or attachable to a garment clip, or configured for securing the hose 11 to a lanyard (not shown), a strap 15i, 15j, harness or head band 19.

An example of the straps 15i, 15j is shown best in FIGS. 1 to 4, which show a first strap 15i extending from a first side of the mask 3 and a second strap 15j extending from a second side of the mask 3. The straps 15i, 15j may extend in opposite directions, such that they are opposing straps 15i, 15j extending from the mask frame 5. However, it will be understood that there may be any number of straps 15i, 15j extending in any suitable direction. The straps 15i, 15j may be flexible. The straps 4 may comprise an elastomer, for example the straps 4 may be made of silicone.

The straps 15i, 15j may extend from the mask frame 5. The straps 15i, 15j may be arranged to extend around part of the patients face. The straps 15i, 15j may be configured to extend around a patient's head entirely, such that the straps 15i, 15j directly attach to each other. The straps 15i, 15j may be joined to each other so that they form a unitary strap extending from opposing ends of the mask 3.

In alternative examples, the straps 15i, 15j may attach to a harness or patient head band 19, as shown in FIG. 3. This allows different sizes of harness or head band 19 to be selected for a given patient, or for an adjustable harness or head band 19 to be selected. The harness or head band 19 may have attachment portions that attach to corresponding attachment portions on the straps 15i, 15j. As shown in FIG. 3, the straps 15i, 15j may include one or more slots 16i, 16j (alternatively referred to as apertures) that provide attachment portions for attaching a patient head band, or clip for attaching a patient head band, although it will be clear that other attachment portions may be provided on the straps 15i, 15j or directly on the mask 3.

The nasal respiratory mask 3 may include means to monitor a carbon dioxide level of the mask 3, and in particular monitor the nasal breathing cavity. The nasal respiratory mask 3 may comprise a carbon dioxide monitoring line connector 31 for attaching a carbon dioxide sensor 30, as shown in FIGS. 1 to 5.

FIG. 5 shows an example of a nasal respiratory mask 3 comprising a carbon dioxide monitoring line 32 extending between the carbon dioxide monitoring line connector 31 and the carbon dioxide sensor 30, although it will be appreciated the carbon dioxide sensor 30 may connect to the carbon dioxide monitoring line connector 31 directly. In the example shown in FIG. 5 the carbon dioxide monitoring line connector 31 is located on a lower end of the mask frame 5, although it will be appreciated that the carbon dioxide monitoring line connector 31 may be located at any suitable position on the mask 3. The carbon dioxide monitoring line connector 31 may include a cap for sealing a port of the carbon dioxide monitoring line connector 31 when not is use, and/or the carbon dioxide monitoring line connector 31 may be self-sealing so as to substantially prevent through-flow across the port when the carbon dioxide monitoring line 32 is disconnected from the carbon dioxide monitoring line connector 31. The carbon dioxide monitoring line 32 may include an elbow connector, and/or may form a swivel connection with the carbon dioxide monitoring line connector 31, as described in relation to hose attachment portion 10 and connector 12.

The carbon dioxide monitoring line 32 may be at least partially, and in some cases entirely, formed from a water permeable material, and function similarly to the permeable material of the mask frame 5 and hose 11 described above in that it allows liquid water and/or water vapour therethrough. The permeable material may be configured to reduce, restrict or prevent flow of gases therethrough. A carbon dioxide monitoring line 32 formed, at least partially, of a permeable material reduces the build-up of water and/or water vapour inside the carbon dioxide monitoring line 32. Similarly, the carbon dioxide monitoring line connector 31 may be at least partially, and in some cases entirely, formed from the water permeable material.

The hose 11 may be malleable, and/or comprise a malleable member 17 (as shown in FIG. 6), such that the hose 11 is able to retain a given shape or position when manipulated (e.g. bent or twisted) into that shape or position. This allows the hose 11 to be positioned so as to improve patient comfort and/or clinician access. The entire length of the hose 11 may be malleable, or comprise a malleable member 17, or only a portion of the hose 11 may be malleable, or comprise a malleable member 17.

The malleable properties of the malleable member 17 mean that it retains a given shape or position when manipulated (e.g. bent or twisted), such that the hose 11 is able to retain a given shape or position when manipulated into that shape or position. This allows the hose 11 to be positioned so as to improve patient comfort and/or clinician access. The malleable member 17 may extend along the entire length of the hose 11, or the malleable member 17 may extend along only a portion of the length of the hose 11 (e.g. 50% of the length). In some examples, the malleable member 17 may extend from the hose 11, i.e. from a location between or at the ends of the hose 11, and attach to or press against an external object (e.g. part of the patient) or part of the nasal respiratory system 2 (e.g. one of the straps 15i, 15j, or the harness/head band 19) so as to support the hose 11 via the object.

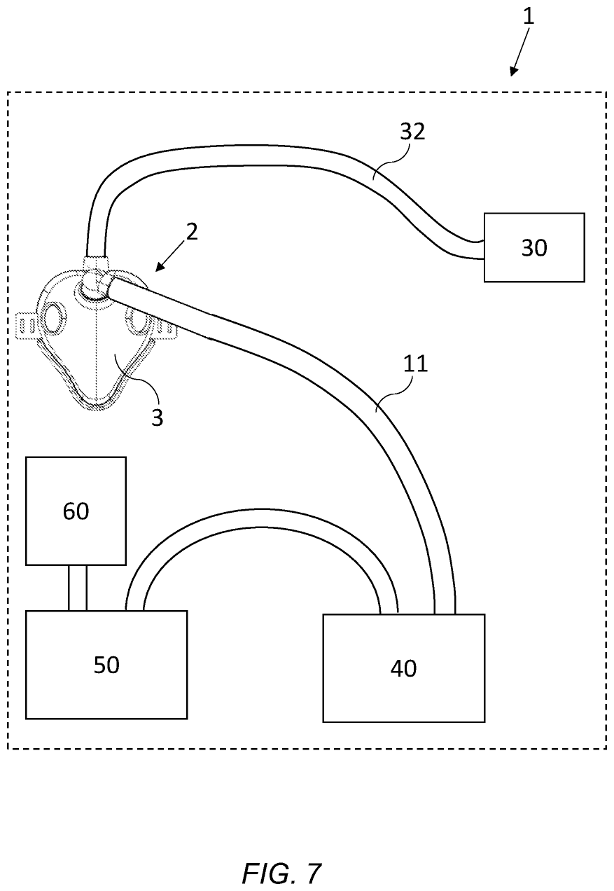

FIG. 7 shows an example of a high flow oxygen therapy apparatus 1 including a nasal respiratory mask system 2.

The high flow oxygen therapy apparatus 1 may include a carbon dioxide sensor 30 connected to the nasal respiratory mask 3, as described above.

The hose 11 may extend from the nasal respiratory mask 3 to a heating chamber and/or humidification chamber 40 that selectively and controllably heats and humidifies the oxygen enriched air supplied to the patient through the nasal respiratory mask.

The heating chamber and/or humidification chamber 40 may be arranged between the mask 3 and a ventilator 50. The ventilator 50 may be arranged to produce the high flow of oxygen enriched air. The oxygen enriched air may be supplied by an oxygen enriched air supply 60 connected to the ventilator 50. In some examples, the carbon dioxide sensor 30 may be integrated into the ventilator 50.

The nasal respiratory mask 3 includes at least one mask aperture providing a gas flow path through the mask between the nasal breathing cavity and ambient air outside the mask. The mask aperture has an adjustable flow restrictor for restricting the flow of gas from the nasal breathing cavity directly to ambient. Adjustment of the adjustable flow restrictor is performed to maintain a positive end-expiratory pressure (PEEP) in the nasal breathing cavity of between 0.2 kPa and 1 kPa during the administering of high flow oxygen therapy to the patient.

The nasal respiratory mask 3 may include at least one mask aperture 20i, 20j. The example shown in FIGS. 1 to 4 is shown to include two mask apertures 20i, 20j although the nasal respiratory mask 3 may comprise any number of mask apertures 20i, 20j, for example one, two, three or four.

In one arrangement, the mask aperture(s) each include a passive one way valve 20i, 20j. The one or more one-way valves 20i, 20j are configured to move from a closed position in which air is restricted from flowing through the one-way valve 20i, 20j into the nasal breathing cavity, to an open position in which air can flow from the nasal breathing cavity through the one-way valve 20i, 20j to outside the mask.

The one-way valves 20i, 20j allow the pressure in the nasal breathing cavity of the mask 3 to drop when it reaches the opening pressure of the valve 20i, 20j, or at least prevents the pressure in the nasal breathing cavity increasing above a given value, e.g. 1 kPa. Above 1 kPa (approximately 10 cmH2O), expiration by the patient into this nasal breathing cavity pressure may become uncomfortable.

When a patient breathes in, the pressure in the nasal breathing cavity remains below the valve opening pressure such that the one-way valve 20i, 20j is arranged in the closed position. When the patient breathes out, the pressure in the nasal breathing cavity will increase and may increase above the valve opening pressure such that the one-way valve 20i, 20j moves from the closed position to the open position. This allows air to escape from the nasal breathing cavity and, in particular, allows expired gases from the patient to be expired when breathing out, whilst preventing ambient air being inhaled by the patient through the one-way valve 20i, 20j when breathing in.

The one-way valve(s) 20i, 20j may have a valve opening pressure of approximately 0.5 kPa (approximately 5 cm H2O), which is expected to be sufficient to wash out gases expired by the patient. The valve opening pressure may be less than 1 kPa (approximately 10 cm H2O). Although it will be appreciated that the valve opening pressure may be any suitable value. The valve opening pressure may be less than 0.8 kPa (approximately 8 cm H2O) or less than 0.6 kPa (approximately 6 cm H2O). The valve opening pressure may be greater than 0.2 kPa (approximately 2 cm H2O), greater than 0.3 kPa (approximately 3 cm H2O) or greater than 0.4 kPa (approximately 4 cm H2O).

Adjustment of the valve opening pressure may enable adjustment of the flow restriction provided by the one-way valve in the mask aperture. The valve opening pressure adjustment may be performed manually, i.e. without and closed loop control of the valve so that the one-way valve remains fully passive in its operation to open and close.

The mask cushion 6 may be arranged to form a seal that is maintained up to pressures that at least match the opening pressure of the valve(s) 20i, 20j, so as to prevent excessive leakages of air below the opening pressure.

The desirable flow rate through the one-way valve 20i, 20j is determined based on ensuring the pressure in the nasal breathing cavity is maintained at a suitable level during expiration of the patient. The flow rate through the one-way valve 20i, 20j in the open position may be at least 2 litres per minute, or preferably at least 5 litres per minute. To prevent excessive pressure loss through the one-way valve 20i, 20j, the flow rate through the one-way valve 20i, 20j in the open position may be less than 20 litres per minute in use.

The one-way valve(s) 20i, 20j defines an opening 21 that extends through the mask frame 5 in the open position, and through which the air can flow from the nasal breathing cavity to outside the mask 3. The opening may have a cross-sectional area of between 0.5 cm² and 15 cm², and preferably between 1 cm² and 8 cm².

The one-way valve(s) 20i, 20j may be any suitable one-way valve.

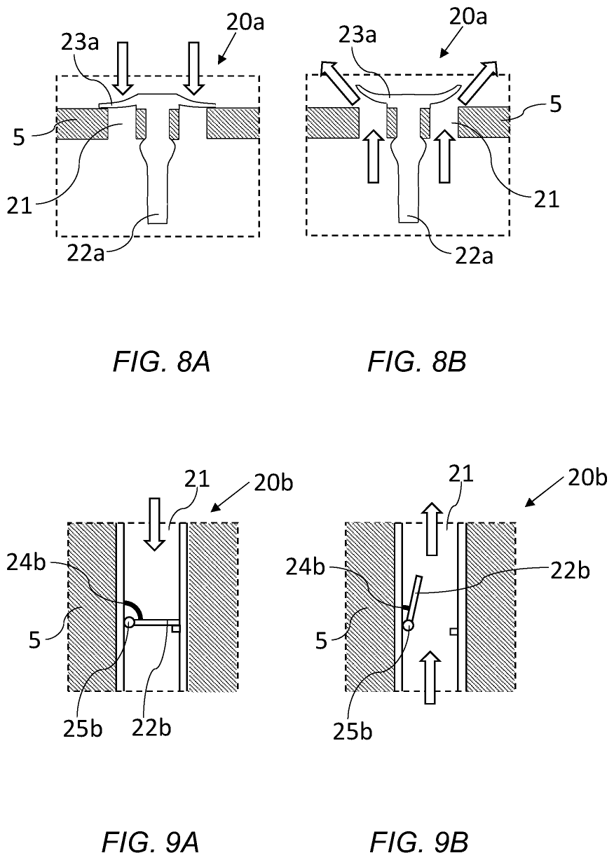

FIGS. 8A and 8B show an example in which the one-way valve 20i, 20j is a one-way umbrella valve 20a. As shown in FIG. 8A, the umbrella valve 20a has a generally umbrella shaped valve member 22a comprising a dome-shaped diaphragm 23a that sits over the opening 21 and prevents air passing through the opening 21, from outside the mask 3 to inside the nasal breathing cavity of the mask 3, when the valve 20a is in the closed position. When the flow of air is reversed, such that when air pressure is applied to the bottom of the diaphragm 23a, the diaphragm is forced upwards at the valve opening pressure so that the diaphragm 23a assumes an upwardly curved shape that allows air to pass through the opening from the nasal breathing cavity to outside the mask 3, as shown in FIG. 8B.

FIGS. 9A and 9B show an example in which the one-way valve 20i, 20j is a flapper valve 20b. The flapper valve 20b comprises a hinge or pivot mechanism 25b that provides limited rotation of the valve member 22b, so that air travelling through the opening 21 from outside the mask 3 to inside the nasal breathing cavity of the mask 3 moves or holds the valve member 22b in a closed position. When the air flow is reversed, the valve member 22b is moveable through an angle range permitted by the hinge 25b, so as to move the valve member 22b from the closed position (FIG. 9A) to the open position (FIG. 9B). The movement of the hinge 25b from the closed position to the open position is restricted, so that the air pressure must reach a minimum (valve opening) pressure. For example, the valve member 22b may have a minimum weight that counters any movement, or the hinge 25b may be spring-loaded by a spring 24b. The hinge 25b may be adjustable so as to adjust the valve opening pressure and/or adjust the angular range of the hinge and thereby adjust the size of the opening through the one-way valve.

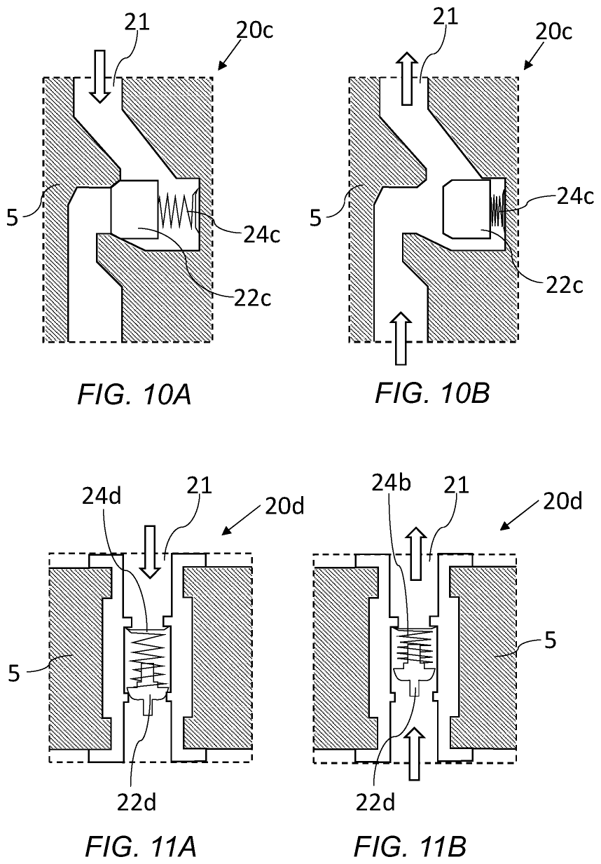

FIGS. 10A and 10B show an example in which the one-way valve 20i, 20j is a lift-check valve 20c. The lift-check valve 20c comprises a valve member 22c biased by a spring 24c against a wall of the mask frame 5, so as to block the opening 21. When air flows from outside the mask 3 through the opening 21 (See FIG. 10A), the valve member 22c remains in a closed position of the valve 20c so as to prevent airflow through the opening 21 into the nasal breathing cavity. When the air flow reverses, the air pressure pushes against the valve member 22c and against the spring 24c so as to move the valve member 22c from the closed position (FIG. 10A) to the open position (FIG. 10B). Air is thereby able to flow from inside the nasal breathing cavity, through the opening 21, and outside the mask 3.

FIGS. 11A and 11B show an example in which the one-way valve 20i, 20j is a spring-loaded inline one-way valve 20d. FIG. 11A shows the valve 20d extending through an opening 21 of the mask frame 5. The valve 20d is in a closed position, such that the valve member 22d prevents air travelling through the opening 21 from outside the mask 3 to inside the nasal breathing cavity of the mask 3. When the air flow is reversed, the air pressure pushes against the valve member 22d so as to move the valve member 22d from the closed position (FIG. 11A) to the open position (FIG. 11B). A spring 24d biases the valve member 22d into the closed position, such that the air pressure must reach a minimum pressure value (i.e. valve opening pressure) to move the valve member 22d against the spring 24d, so as move to the open position and allow air to move through the opening 21.

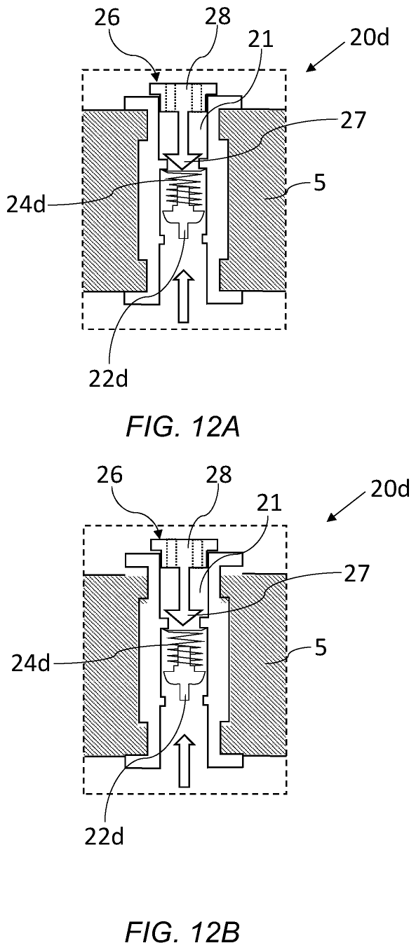

The spring 24d may be selected to provide a particular valve opening pressure, and/or be adjustable so as to vary the valve opening pressure. Where the valve opening pressure is not adjustable, a separate adjustable flow restrictor may be provided in line with the one-way valve. An adjustable flow restrictor 26 configured to adjust a minimum size of the opening 21 on one side of the one-way valve 20d. For example, FIGS. 12A and 12B show an adjustable flow restrictor 26 comprising a wedge-shaped member 27 that engages a neck of the opening 21. Rotation of the head 28 of the adjustable flow restrictor 26 varies an air gap between the wedge-shaped member 27 and the neck of the opening 21 so as to control the minimum size of the opening 21. FIG. 12A shows the adjustable flow restrictor 26 in a first position, defining a first minimum size of the opening 21, and FIG. 12B shows the adjustable flow restrictor 26 in a second position, defining a second minimum size of the opening 21 that is greater than the minimum size of the opening 21 shown in FIG. 12A. This allows the flow rate through the one-way valve 20d to be variable. It will be understood that any of the above-mentioned one-way valves 20i, 20j, 20a, 20b, 20c, 20d may comprise an adjustable flow restrictor 26 configured to adjust a minimum size of the opening 21 so as to adjust the flow rate through the one-way valve 20i, 20j, 20a, 20b, 20c, 20d.

It will be understood that in some examples, the valve 20i, 20j, 20a, 20b, 20c, 20d opening and closing pressures may differ slightly due to static friction and other effects causing some hysteresis.

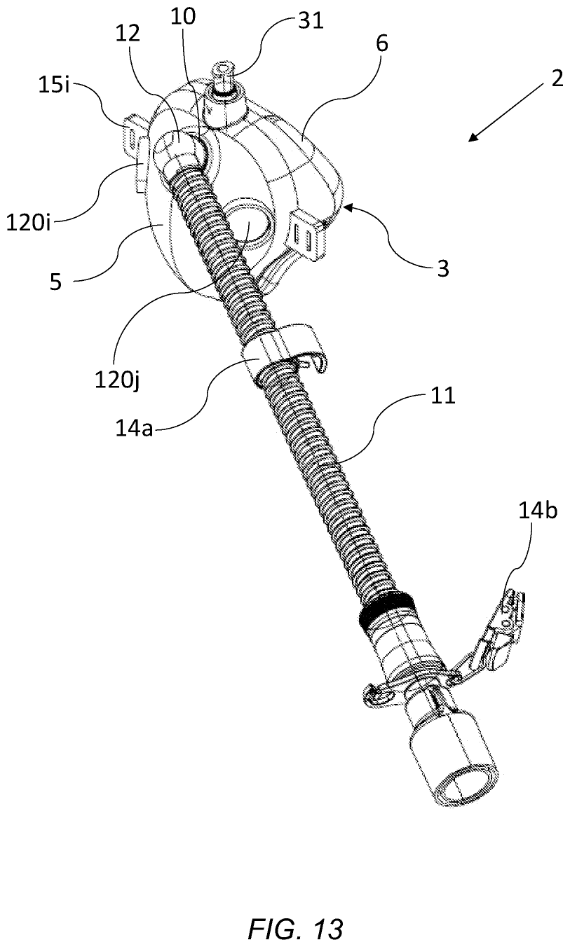

In some examples, as shown in FIG. 13, the nasal mask 3 may include one or more mask apertures with one-way valves 120i, 120j whose movement from the closed position to the open position is entirely dependent on the pressure in the nasal breathing cavity of the mask 3, such that the one-way valves do not have a minimum opening pressure. In other words, the valve member may be substantially unbiased, with movement from the closed position to the open position dependent on the pressure in the nasal breathing cavity. It will be appreciated that the one-way valve may have some relative bias due to friction or the weight of the valve member, but that this bias is minimised for all practical purposes. Examples of such unbiased valves will now be described.

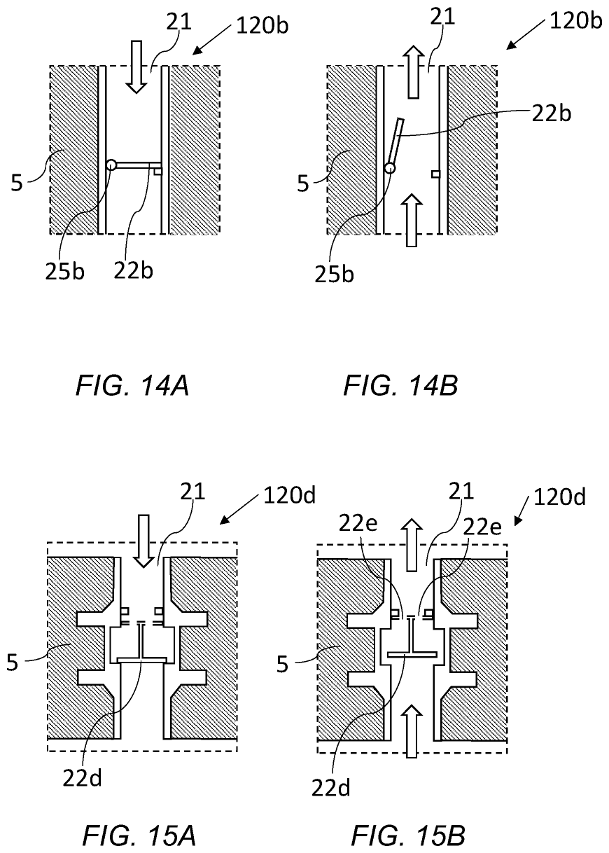

FIGS. 14A and 14B show an example in which the one-way valve is an unbiased flapper valve 120b similar to that shown in FIGS. 9A and 9B except it does not have a spring 24b. The flapper valve 120b comprises a hinge or pivot mechanism 25b that provides limited rotation of the valve member 22b, so that air travelling through the opening 21 from outside the mask 3 to inside the nasal breathing cavity of the mask 3 moves the valve member 22b into a closed position. When the air flow is reversed, the valve member 22b is moveable through a permitted angle range, so as to move the valve member 22b from the closed position (FIG. 14A) to the open position (FIG. 14B).

FIGS. 15A and 15B show an example in which the one-way valve is an unbiased inline one-way valve 120d. FIG. 15A shows the valve 20d extending through an opening 21 of the mask frame 5. The valve 120d is in a closed position, such that the valve member 22d prevents air travelling through the opening 21 from outside the mask 3 to inside the nasal breathing cavity of the mask 3. When the air flow is reversed, the air pressure pushes against the valve member 22d so as to move the valve member 22d from the closed position (FIG. 15A) to the open position (FIG. 15B), allowing air to flow around the valve member and through apertures 22e.

The one-way valves of FIGS. 14A, B and 15A, B are provided in line with an adjustable flow restrictor 26 configured to adjust a minimum size of the opening 21 on one side of the one-way valve, such as shown in FIGS. 12A and 12B. The adjustable flow restrictor 26 may be compatible with any suitable one-way valve, such as the one-way valves 120b, 120d described above. The adjustment of the adjustable flow restrictor 26 may be performed manually.

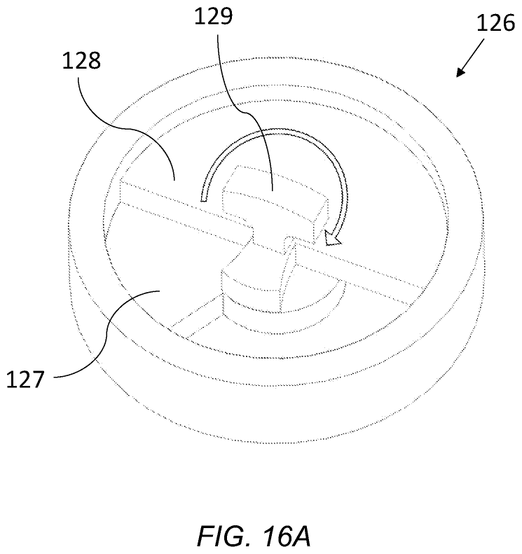

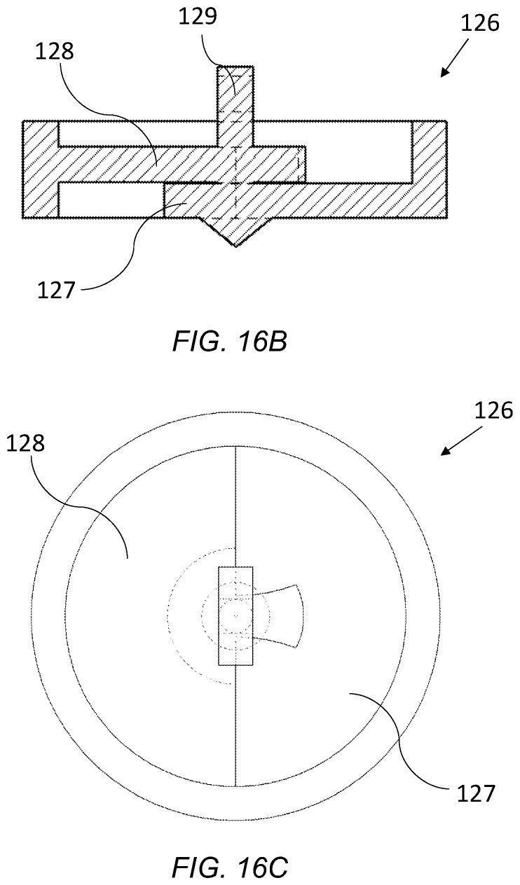

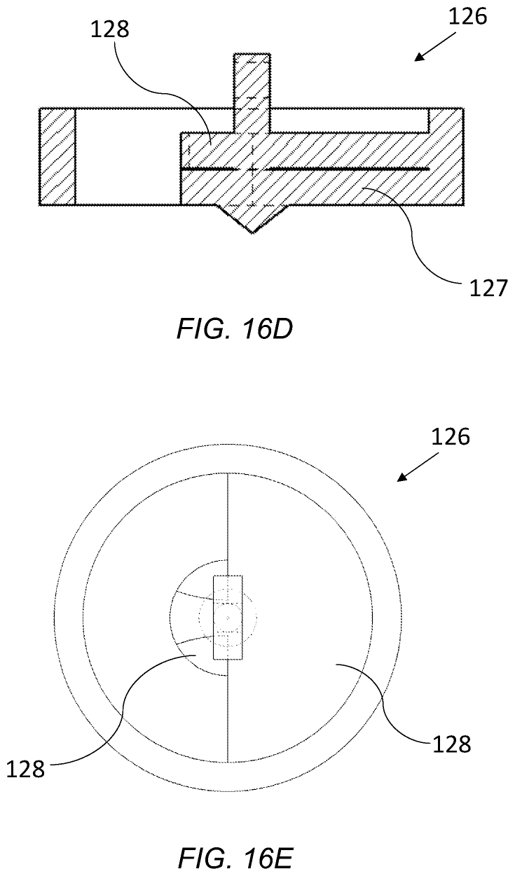

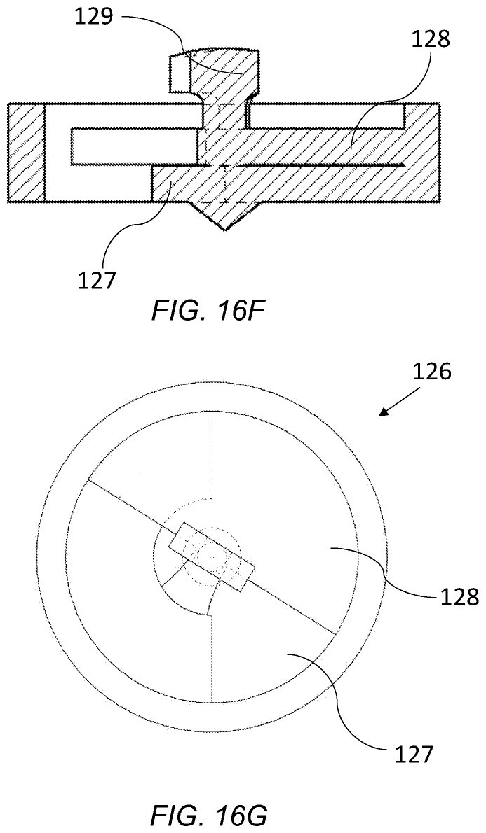

FIGS. 16A to 16G show a further example of an adjustable flow restrictor 126 that can be placed in or over the opening 21 to be used in line with either an open vent or a passive one-way valve, as described above. The adjustable flow restrictor 126 comprising a moveable element 128 that rotates about an axis relative to a fixed element 127 of the adjustable flow restrictor 127. Rotation of the moveable element 128 (e.g. via a handle 129) adjusts an opening through the adjustable flow restrictor 126 through which air can flow. In this way, the effective size of the opening 21 is variable.

FIGS. 16B and 16C show the adjustable flow restrictor 126 in a closed position, in which the moveable element 128 has been rotated to entirely cover the opening and prevent air flow therethrough.

FIGS. 16D and 16E show the adjustable flow restrictor 126 in an open position, in which the moveable element 128 has been rotated to entirely uncover the opening and allow air flow therethrough. In this particular example, the moveable element 128 is rotated by approximately 180 degrees with respect to its position in the closed position of FIGS. 16B and 16C, however it will be appreciated that the relative degrees of rotation to cover and uncover the opening may vary.

FIGS. 16F and 16G show the adjustable flow restrictor 126 in a partially open position, in which the moveable element 128 has been rotated to partially cover the opening and allow some relative air flow therethrough.

As will be apparent, the position of the moveable element 127 can be varied to control the effective size (cross sectional area) of the opening 21. In the example of FIGS. 16A to 16G, the moveable element 128 is rotated between 0 and 180 degrees to vary the degree of the opening that is uncovered, although any suitable range may be provided.

The adjustable flow restrictor 26, 126 can be tuned so as to adjust the size of the opening on one side of the one-way valve. In this way the nasal breathing cavity can be maintained at a selectable positive pressure value, for instance a positive pressure of between 0.2 kPa and 1 kPa can be maintained. This allows the mask 3 to be used at a range of different HFOT supply flow rates to meet or slightly exceed the peak inspiratory flow rate of the patient, whilst maintaining the positive pressure within the nasal breathing cavity to within an effective range. The effective range ensures the positive pressure is not too high so as to be uncomfortable for the patient and not too low so as to be ineffective at maintain the nasal airways open for gas recruitment to the lungs. A positive pressure above 1 kPa is considered uncomfortable for a patient, whilst a positive pressure below 0.2 kPa reduces the effectiveness of the treatment. The positive pressure may be maintained at a level between 0.4 kPa and 0.8 kPa, or at approximately 0.5 kPa.

The mask apertures may be positioned in an optimal position for allowing expiration from the nasal respiratory mask 3, whilst minimising loss of the oxygen enriched air fed through the hose 11. Nasal respiratory masks 3 comprising two or more mask apertures may arrange the apertures 20i, 20j to be in a spaced arrangement across the mask frame 5. As shown best in FIG. 2, the mask apertures may be spaced substantially symmetrically about a vertical centre line of the mask frame 5. Substantially in this context refers to allowing for manufacturing tolerances. This allows the mask apertures to be arranged with respect to the generally symmetrical face of a patient. For example, the mask apertures may be located towards a lower end of the mask frame 5 so as to be adjacent either side of a user's mouth when worn. The mask apertures may be located on a vector of the nostril, so that air from the nasal passages is directed to the mask apertures.

In some examples, the nasal respiratory mask 3, and particularly the mask frame 5, may have no gas flow apertures other than the hose attachment portion 10, the carbon dioxide monitoring line connector 31, and the mask apertures. This helps to ensure the gas flow into and out of the nasal respiratory mask 3 is controlled.

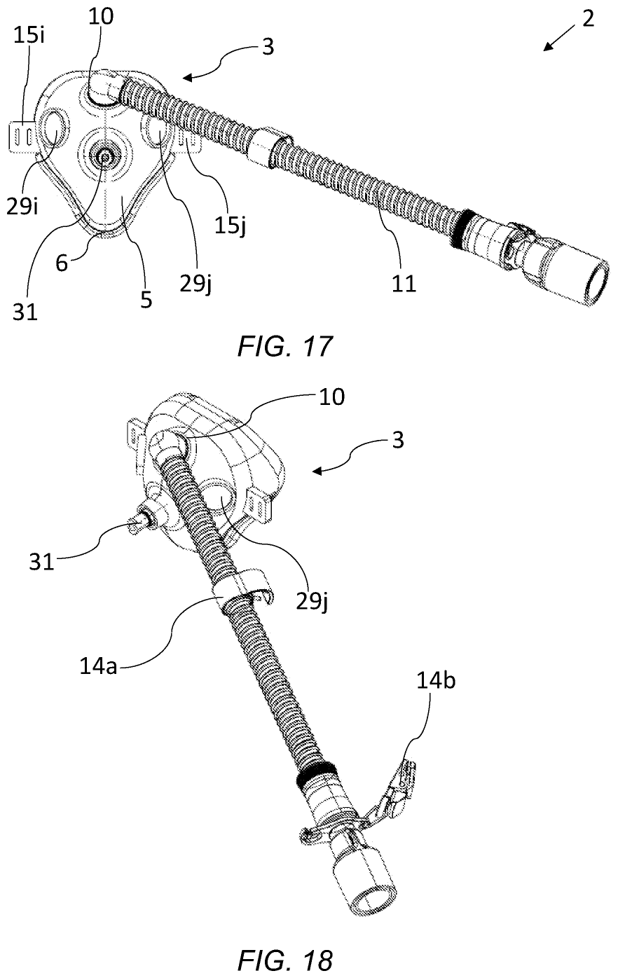

FIGS. 17 and 18 show a nasal respiratory mask system 2 according to a second example. The second example is substantially the same as the first example of a nasal respiratory mask system 2 shown in FIGS. 1 to 6, except that the valves 20i, 20j are replaced by vents 29i, 29j and the carbon dioxide monitoring line connector 31 is located substantially in the middle of the mask frame 5. It will be appreciated that the carbon dioxide monitoring line connector 31 and hose attachment portion 10 may be located at any suitable position on the nasal respiratory mask 3, for example, FIGS. 17 and 18 show the carbon dioxide monitoring line connector 31 above the hose attachment portion 10 (above in this context referring to the orientation of the mask 3 when on the patient's face).

In some examples, the carbon dioxide monitoring line connector 31 and hose attachment portion 10 may be one component on the mask 3. For example, a single connector located on the mask 3 may comprise, or function as, the carbon dioxide monitoring line connector 31 and hose attachment portion 10.

The vents 29i, 29j operate substantially the same as the valves 20i, 20j in the open position of the previous examples, in that they each define an opening/aperture through the mask 3. In order to ensure an increased pressure is maintained within the mask 3 and delivered to the patient, the vents 29i, 29j each define an opening having a total cross-sectional area (i.e. the opening of each vent 29i, 29j combined) less than a cross-sectional area of the opening through the hose attachment portion 10. The total cross-sectional area may be less than 80% of the cross-sectional area of the opening through the hose attachment portion 10, and in some examples less than 50%.

In contrast to the one-way valves 20i, 20j, 120i, 120j of the previous examples, the vents 29i, 29j remain in an open position so that air can flow from the nasal breathing cavity through each vent 29i, 29j to outside the mask 3 at all times, e.g. during both inhalation and exhalation of the patient. However, the extent to which air flow can travel through the vent 29i, 29j may be variable by using an adjustable flow restrictor 26, 126, such as described above.

In particular, the adjustable flow restrictor 26, 126 can be tuned so as to adjust the size of the opening through the vent 29i, 29j. In this way the nasal breathing cavity can be maintained at a selectable positive pressure value, for instance a positive pressure of between 0.2 kPa and 1 kPa can be maintained. This allows the mask 3 to be used at a range of different flow rates to meet or slightly exceed the peak inspiratory flow rate of the patient, whilst maintaining the positive pressure within the nasal breathing cavity to within an effective range. The effective range ensures the positive pressure is not too high so as to be uncomfortable for the patient and not too low so as to be ineffective at maintain the nasal airways open for gas recruitment to the lungs. A positive pressure above 1 kPa is considered uncomfortable for a patient, whilst a positive pressure below 0.2 kPa reduces the effectiveness of the treatment and may result in entrainment of ambient air through the vent 29i, 29j. The positive pressure may be maintained at a level between 0.4 kPa and 0.8 kPa, or at approximately 0.5 kPa.

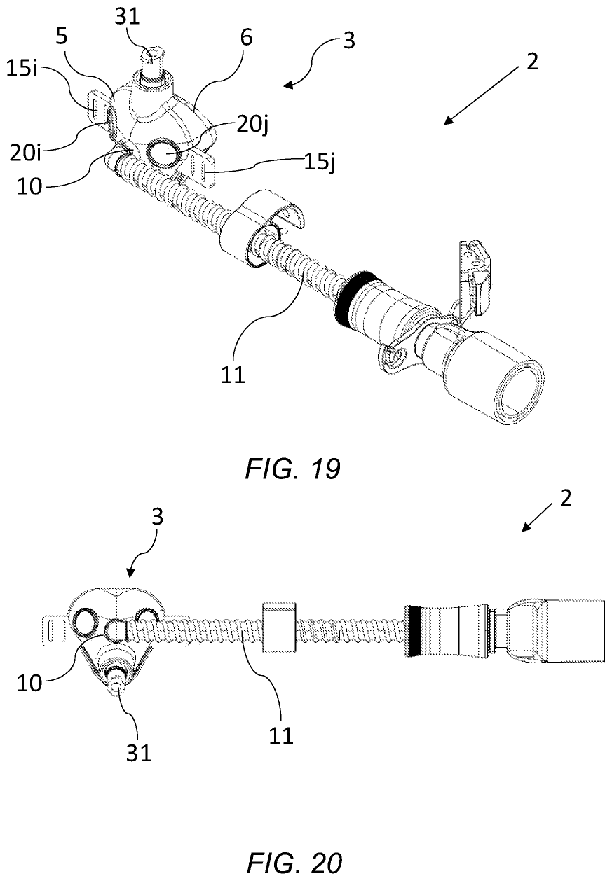

FIG. 19 shows a neo-natal nasal respiratory mask system 2 according to a third example. The third example is substantially the same as the first example of a nasal respiratory mask system 2 shown in FIGS. 1 to 7, except that the nasal respiratory mask 3 is for neo-natal patients. The size of the nasal respiratory mask 3 of the third example is therefore tailored to dimensions suitable for neo-natal patients.

In particular, the nasal respiratory mask 3 may be smaller so as to extend over the face of the neo-natal patient, thereby covering the nasal passages, without extending over the eyes or entirely over the face of the patient. This ensures an adequate seal of the mask 3 to the face of the patient is formed, whilst also maintaining patient comfort.

Other features of the neo-natal nasal respiratory mask system 2 may also be smaller with respect to those features of the adult nasal respiratory mask system 2 of the first and second examples. For example, the size of the straps 15i, 15j. The length and/or diameter of the hose 11 may be smaller than the length and/or diameter of the hose 11 of an adult nasal respiratory mask system 2.

Alternatively, many of the features may be the same size as for the adult nasal respiratory mask system 2. For example, the carbon dioxide monitoring line connector 31 and hose attachment portion 10 may be a standard size, thereby allowing a single carbon dioxide monitoring line 32 or hose 11 to connect to any of the nasal respiratory masks 3.

In the example shown in FIG. 19, the hose attachment portion 10 is positioned towards a middle of the mask 3. However, it will be understood the hose attachment portion 10 may be positioned in any suitable position on the mask 3.

In some examples of a neo-natal nasal respiratory mask system 2, the flow rates supplied through the hose 11 and to the patient may be less than that required for an adult patient. For example, in order to approximately match or exceed the neo-natal patient's peak inspiratory flow rates, the flow rate delivered to the patient may be lower than 20 LPM, however it is generally at least 5 LPM.

Accordingly, the valve opening pressure may be similarly adjusted and/or adjustable to suit the needs of a neo-natal patient. The one-way valve(s) 20i, 20j may have a valve opening pressure of approximately 0.5 kPa (approximately 5 cm H2O), or a lower value of approximately 0.3 kPa (approximately 3 cm H2O). In other examples, the one-way valve 120i, 120j may be unbiased, such as described in relation to FIGS. 13 to 15.

It will be understood that the mask 3 may include one-way valves 20i, 20j, 120i, 120j and/or vents 29i, 29j, as required. The flow rate through the one-way valves 20i, 20j or vents 29i, 29j may be at least 1 litres per minute, and is preferably at least 3 litres per minute or 5 litres per minute. The flow rate may be approximately linked to the patient's weight, e.g. 2 litres per minute per kg.

In some examples, the mask frame 5 and mask cushion 6 may be integrally formed of a single material, thereby allowing the neo-natal nasal respiratory mask 3 to be lighter for the patient. For example, the neo-natal nasal respiratory mask 3 may be formed of silicone. Alternatively, the construction of the neo-natal nasal respiratory mask 3 may be the same as for the adult nasal respiratory mask 3 described in relation to the first and second examples.

FIG. 20 shows a neo-natal nasal respiratory mask system 2 according to a fourth example. The fourth example is substantially the same as the third example of a nasal respiratory mask system 2 shown in FIG. 19, except that the carbon dioxide monitoring line connector 31 is located substantially in the middle of the mask 3 above the hose attachment portion 10, such as is shown and described in relation to the second example of FIGS. 17 and 18.

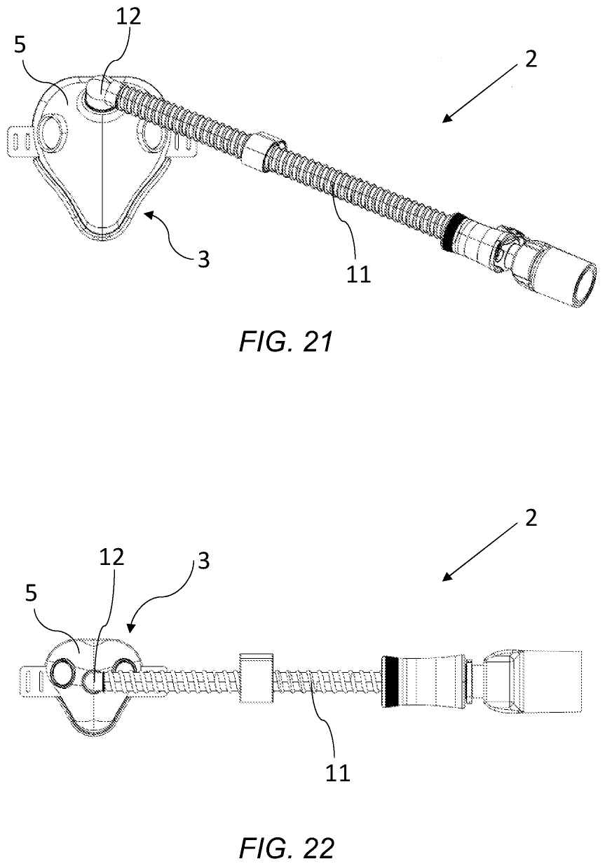

In some examples, the mask 3 and/or mask frame 5 may not include a carbon dioxide monitoring line connector 31. FIG. 21 shows an example of an adult nasal respiratory mask 3 that does not include a carbon dioxide monitoring line connector 31 and FIG. 22 shows an example of a neo-natal nasal respiratory mask 3 that does not include a carbon dioxide monitoring line connector 31.

In some examples, a carbon dioxide monitoring line connector 31 may be attached to the hose 11 or other component of the mask 3, e.g. connector 12. The carbon dioxide monitoring line connector 31 may be arranged to substantially prevent air flow therethrough when a carbon dioxide monitoring line 32 is not coupled to the carbon dioxide monitoring line connector 31. For example, the carbon dioxide monitoring line connector 31 may be a pneumatic quick connect coupling or similar, or a connector cap (not shown) may be selectively placed over the connector 31.

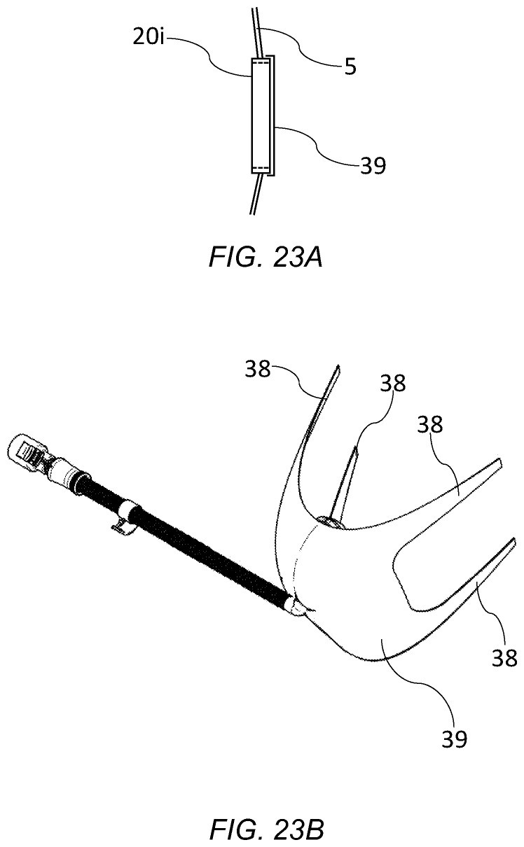

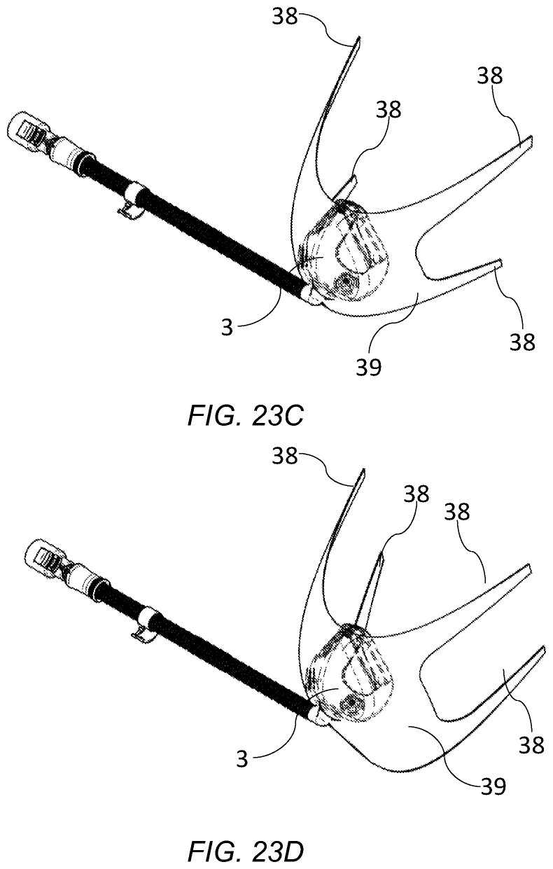

In some examples, the one-way valves 20i, 20j and/or vents 29i, 29j may include a filter membrane 39 covering the opening/aperture of the one-way valves 20i, 20j and/or vents 29i, 29j, for example as shown in FIG. 23A. The filter membrane 39 may be located against an inner surface of the mask frame 5, an outer surface of the mask frame 5, or in the opening between the inner and outer surfaces. The filter membrane 39 may be arranged to reduce the spread of contaminants from the patient's exhaled breath. In some examples the filter membrane 39 may extend over a significant portion (i.e. over half) of the mask frame 5 and/or extend to cover the patient's mouth.

FIGS. 23B and 23C show an example in which the filter membrane 39 is configured to cover the whole of the mask frame 5. In some examples, the filter membrane 39 may include straps 38 to assist in attaching the filter membrane 39 to the patient or mask 3. One or more straps 38 may extend from the filter membrane 39. As shown in FIG. 23B, the straps 38 may be elongate strips arranged to be tied together or to an external object, such as part of the nasal respiratory mask system 2. Alternatively, the straps 38 may comprise a fastener on an end of the strap 38 for fastening to an adjacent strap 38 or external object. The straps 38 may be arranged as ear straps, which wrap around an ear, or head straps, which wrap around the patient's head. The straps 38 may be formed of the same material as the filter membrane 39, or an alternative material. The straps 38 may be elastic or substantially rigid. Alternatively or in addition, the filter membrane 39 may include an elastic strip (not shown) extending circumferentially around the filter membrane 39 to fit over the patient's head.

In some examples, the filter membrane 39 may be arranged to also cover the patient's mouth. FIG. 23D shows an example substantially the same as the example shown in FIGS. 23B and 23C, except the filter membrane 39 extends over the patient's mouth in addition to extending over the mask frame 5.

A filter membrane 39 covering at least part the mask frame 5, as well as optionally the patient's mouth, can help to reduce the spread of particles from the patient's exhaled breath to the environment, increasing safety for clinicians and other patients.

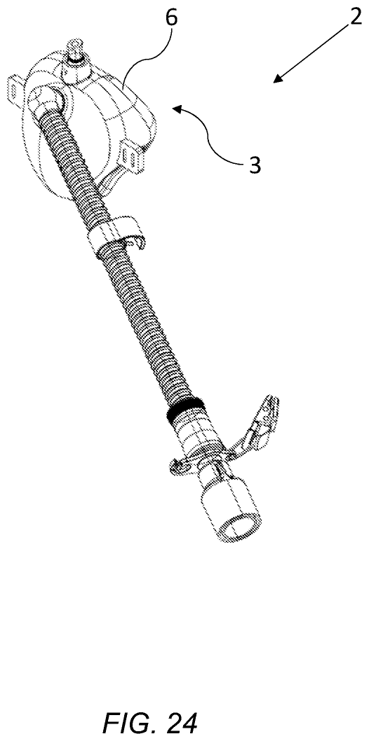

In some examples, the nasal respiratory mask system 2 may not include a one-way valve or vent. FIG. 24 shows an example of an adult nasal respiratory mask 3, arranged to cover the nasal passages but not the mouth, which does not include a one-way valve. The mask cushion 6 may be arranged to form a sufficient seal to prevent excessive leaks, but will not be fully sealed to the patient's face, for example leaks that may detrimentally lower the flow rate, thereby causing poor performance, but will typically leak more than the nasal respiratory mask systems 2 of FIGS. 1 to 23 during patient outflow, due to the absence of a one-way valve to evacuate excess air pressure. The leakages from the mask 3 and mask cushion 6 may be designed so as to substantially direct flow away from certain parts of the patient, such as the patient's eye, thereby improving patient comfort. For example, the mask cushion 6 may be contoured to form a tighter seal at an upper half of the mask cushion 6, adjacent the patient's eyes, than at a lower half of the mask cushion 6, adjacent the patient's mouth.

The masks 3 described above may be configured to engage the patient's nose without the use of nasal prongs that enter the patient's nasal passages. This may improve patient comfort, whilst still allowing the delivery of high flow rates of air to the patient.

Where the word ‘or’ appears this is to be construed to mean ‘and/or’ such that items referred to are not necessarily mutually exclusive and may be used in any appropriate combination.

Although the invention has been described above with reference to one or more preferred embodiments, it will be appreciated that various changes or modifications may be made without departing from the scope of the invention as defined in the appended claims.

1. A method of administering high flow oxygen therapy to a patient using a high flow oxygen therapy apparatus, the apparatus comprising:
a nasal respiratory mask comprising a mask frame; a mask cushion on the mask frame, the mask frame and mask cushion defining a nasal breathing cavity; a hose attachment portion; and a mask aperture having an adjustable flow restrictor for restricting the flow of gas from the nasal breathing cavity directly to ambient; and
an oxygen enriched air supply having a variable flow rate output for supplying heated, humidified oxygen enriched air to the patient,
the method comprising:
providing the nasal respiratory mask to the patient's face so that the nasal breathing cavity surrounds the patient's nose and not the mouth, and the mask cushion contacts and substantially seals against the face of the patient;
connecting a hose between the hose attachment portion of the mask and the oxygen enriched air supply;
adjusting the variable flow rate output of the oxygen enriched air supply to provide a flow rate of the heated, humidified oxygen enriched air to the mask which just exceeds the patient's peak nasal inspiratory flow rate;
adjusting the adjustable flow restrictor of the mask to maintain a positive end-expiratory pressure (PEEP) in the nasal breathing cavity of between 0.2 kPa and 1 kPa during the administering of high flow oxygen therapy to the patient.

2. The method of claim 1, further comprising adjusting the adjustable flow restrictor of the mask to maintain a positive end-expiratory pressure (PEEP) in the nasal breathing cavity of approximately 0.5 kPa during the administering of high flow oxygen therapy to the patient, preferably 0.3 to 0.7 kPa, preferably 0.4 to 0.6 kPa.

3. The method according to claim 1,
wherein the mask aperture further comprises a passive one-way valve configured to move from a closed position in which air is restricted from flowing through the one-way valve, to an open position in which air can flow from the nasal breathing cavity through the one-way valve towards the flow restrictor and to outside the mask.

4. The method of claim 3, wherein the one-way valve is a biased one-way valve having a valve opening pressure of less than 1 kPa or less than 0.8 kPa and/or wherein the valve opening pressure is greater than 0.2 kPa.

5. The method of claim 1, wherein a flow rate through the flow restrictor of the mask is adjusted to be at least 2 litres per minute, preferably at least 5 litres per minute.

6. A method of administering high flow oxygen therapy to a patient using a high flow oxygen therapy system, the system comprising:
a plurality of nasal respiratory masks, each mask comprising: a mask frame; a mask cushion on the mask frame, the mask frame and mask cushion defining a nasal breathing cavity; a hose attachment portion; and a mask aperture for restricting the flow of gas from the nasal breathing cavity directly to ambient, each of the plurality of masks having a differently sized mask aperture for restricting the flow of gas from the nasal breathing cavity directly to ambient by a different amount; and
an oxygen enriched air supply having a variable flow rate output for supplying heated, humidified oxygen enriched air to the patient,
the method comprising:
selecting one of the plurality of nasal respiratory masks;
providing the selected nasal respiratory mask to the patient's face so that the nasal breathing cavity surrounds the patient's nose and not the mouth, and the mask cushion contacts and substantially seals against the face of the patient;
connecting a hose between the hose attachment portion of the mask and the oxygen enriched air supply;
adjusting the variable flow rate output of the oxygen enriched air supply to provide a flow rate of the heated, humidified oxygen enriched air to the mask which just exceeds the patient's peak nasal inspiratory flow rate;
wherein the selected nasal respiratory mask is selected to maintain a positive end-expiratory pressure (PEEP) in the nasal breathing cavity of between 0.2 kPa and 1 kPa during the administering of high flow oxygen therapy to the patient.

7. The method according to claim 6, wherein the mask aperture of each mask further comprises a passive one-way valve configured to move from a closed position in which air is restricted from flowing through the one-way valve, to an open position in which air can flow from the nasal breathing cavity through the one-way valve towards the flow restrictor and to outside the mask.

8. The method of claim 7, wherein the one-way valve has a valve opening pressure of less than 1 kPa.

9. The method of claim 6, wherein the mask aperture of each mask has an adjustable flow restrictor; and the method further comprises adjusting the adjustable flow restrictor for restricting the flow of gas from the nasal breathing cavity directly to ambient.

10. The method of claim 9, wherein a flow rate through the flow restrictor of the mask is adjusted to be at least 2 litres per minute, preferably at least 5 litres per minute.

11. A method of administering high flow oxygen therapy to a patient using a high flow oxygen therapy system, the system comprising:
a plurality of nasal respiratory masks, each mask comprising: a mask frame; a mask cushion on the mask frame, the mask frame and mask cushion defining a nasal breathing cavity; a hose attachment portion; and a mask aperture, wherein the mask aperture has a passive one-way valve configured to move from a closed position in which air is restricted from flowing through the one-way valve, to an open position in which air can flow from the nasal breathing cavity through the one-way valve directly to ambient, each of the plurality of masks having a different one-way valve with a different pre-set valve opening pressure of less than 1 kPa; and
an oxygen enriched air supply having a variable flow rate output for supplying heated, humidified oxygen enriched air to the patient,
the method comprising:
selecting one of the plurality of nasal respiratory masks;
providing the selected nasal respiratory mask to the patient's face so that the nasal breathing cavity surrounds the patient's nose and not the mouth, and the mask cushion contacts and substantially seals against the face of the patient;
connecting a hose between the hose attachment portion of the mask and the oxygen enriched air supply;
adjusting the variable flow rate output of the oxygen enriched air supply to provide a flow rate of the heated, humidified oxygen enriched air to the mask;
wherein the selected nasal respiratory mask is selected to maintain a positive end-expiratory pressure (PEEP) in the nasal breathing cavity of between 0.2 kPa and 1 kPa during the administering of high flow oxygen therapy to the patient.

12. The method of claim 11, wherein the mask aperture of each mask has an adjustable flow restrictor; and the method further comprises adjusting the adjustable flow restrictor for restricting the flow of gas from the nasal breathing cavity directly to ambient.

13. The method of claim 12, further comprising adjusting the adjustable flow restrictor of the mask to maintain a positive end-expiratory pressure (PEEP) in the nasal breathing cavity of approximately 0.5 kPa during the administering of high flow oxygen therapy to the patient, preferably 0.3 to 0.7 kPa, preferably 0.4 to 0.6 kPa.

14. The method of claim 11 wherein a flow rate through the flow restrictor of the mask is adjusted to be at least 2 litres per minute, preferably at least 5 litres per minute.

15. A method of administering high flow oxygen therapy to a patient using a high flow oxygen therapy system, the system comprising:
a nasal respiratory mask comprising: a mask frame; a mask cushion on the mask frame, the mask frame and mask cushion defining a nasal breathing cavity; a hose attachment portion; and a mask aperture, wherein the mask aperture has a passive one-way valve configured to move from a closed position in which air is restricted from flowing through the one-way valve, to an open position in which air can flow from the nasal breathing cavity through the one-way valve directly to ambient, wherein the one-way valve has a variable valve opening pressure; and
an oxygen enriched air supply having a variable flow rate output for supplying heated, humidified oxygen enriched air to the patient,
the method comprising:
selecting one of the plurality of nasal respiratory masks;
providing the selected nasal respiratory mask to the patient's face so that the nasal breathing cavity surrounds the patient's nose and not the mouth, and the mask cushion contacts and substantially seals against the face of the patient;
connecting a hose between the hose attachment portion of the mask and the oxygen enriched air supply;
adjusting the variable flow rate output of the oxygen enriched air supply to provide a flow rate of the heated, humidified oxygen enriched air to the mask;
adjusting the valve opening pressure of the mask to maintain a positive end-expiratory pressure (PEEP) in the nasal breathing cavity of between 0.2 kPa and 1 kPa during the administering of high flow oxygen therapy to the patient.

16. The method of claim 15, wherein the one-way valve has a variable valve opening pressure of between 0.0 kPa and 1 kPa.

17. The method of claim 16, further comprising adjusting the valve opening pressure of the mask to maintain a positive end-expiratory pressure (PEEP) in the nasal breathing cavity of approximately 0.5 kPa during the administering of high flow oxygen therapy to the patient, preferably 0.3 to 0.7 kPa, preferably 0.4 to 0.6 kPa.

18. The method of claim 15, wherein the mask aperture has an adjustable flow restrictor; and the method further comprises adjusting the adjustable flow restrictor for restricting the flow of gas from the nasal breathing cavity directly to ambient.

19. The method of claim 18, wherein a flow rate through the flow restrictor of the mask is adjusted to be at least 2 litres per minute, preferably at least 5 litres per minute.

20. A nasal respiratory mask for a high flow oxygen therapy apparatus, comprising:
a mask frame;
a mask cushion on the mask frame for contacting and substantially sealing against a face of a patient, the mask frame and mask cushion defining a nasal breathing cavity;
the mask frame having:
a hose attachment portion for attaching a hose for delivering a supply of oxygen enriched air to the patient; and
a mask aperture having an adjustable flow restrictor for restricting the flow of gas from the nasal breathing cavity directly to ambient,
wherein the adjustable flow restrictor is adjustable to maintain a positive end-expiratory pressure (PEEP) in the nasal breathing cavity of between 0.2 kPa and 1 kPa during the administering of high flow oxygen therapy to the patient.

21. The nasal respiratory mask of claim 20, wherein the mask aperture further comprises a passive one-way valve configured to move from a closed position in which air is restricted from flowing through the one-way valve, to an open position in which air can flow from the nasal breathing cavity through the one-way valve towards the flow restrictor and to outside the mask.

22. The nasal respiratory mask of claim 21, wherein the one-way valve is a flapper valve or a lift-check valve.

23. The nasal respiratory mask of claim 21, wherein the one-way valve has a valve opening pressure of between 0.0 kPa and 1 kPa.

24. A nasal respiratory mask for a high flow oxygen therapy apparatus, comprising:
a mask frame;
a mask cushion on the mask frame for contacting and substantially sealing against a face of a patient, the mask frame and mask cushion defining a nasal breathing cavity;
the mask frame having:
a hose attachment portion for attaching a hose for delivering a supply of oxygen enriched air to the patient; and
a mask aperture having a passive one-way valve configured to move from a closed position in which air is restricted from flowing through the one-way valve, to an open position in which air can flow from the nasal breathing cavity through the one-way valve directly to ambient outside the mask, wherein the one-way valve has a valve opening pressure for maintaining a positive end-expiratory pressure (PEEP) in the nasal breathing cavity of between 0.2 kPa and 1 kPa during the administering of high flow oxygen therapy to the patient.

25. The nasal respiratory mask according to claim 24, wherein the one-way valve is a biased one-way valve having a valve opening pressure of less than 1 kPa or less than 0.8 kPa and/or wherein the valve opening pressure is greater than 0.2 kPa.

26. The nasal respiratory mask of claim 25, wherein the one-way valve is a flapper valve or a lift-check valve.

27. The nasal respiratory mask according to claim 24, wherein the mask aperture further comprises an adjustable flow restrictor for restricting the flow of gas from the nasal breathing cavity directly to ambient, wherein the adjustable flow restrictor is adjustable to maintain the positive end-expiratory pressure (PEEP) in the nasal breathing cavity of between 0.2 kPa and 1 kPa during the administering of high flow oxygen therapy to the patient.

28. The nasal respiratory mask according to claim 27, wherein a flow rate through the flow restrictor of the mask is adjusted to be at least 2 litres per minute, preferably at least 5 litres per minute.

29. A nasal respiratory mask for a high flow oxygen therapy apparatus, comprising:
a mask frame;
a mask cushion on the mask frame for contacting and substantially sealing against a face of a patient, the mask frame and mask cushion defining a nasal breathing cavity;
the mask frame having:
a hose attachment portion for attaching a hose for delivering a supply of oxygen enriched air to the patient; and
a mask aperture for restricting the flow of gas from the nasal breathing cavity directly to ambient, wherein the mask aperture is dimensioned for maintaining a positive end-expiratory pressure (PEEP) in the nasal breathing cavity of between 0.2 kPa and 1 kPa during the administering of high flow oxygen therapy to the patient.

30. The nasal respiratory mask of claim 29, wherein the mask aperture further comprises a passive one-way valve configured to move from a closed position in which air is restricted from flowing through the one-way valve, to an open position in which air can flow from the nasal breathing cavity through the one-way valve towards the flow restrictor and to outside the mask.

31. The nasal respiratory mask of claim 30, wherein the one-way valve is a flapper valve or a lift-check valve.

32. The nasal respiratory mask according to claim 29, wherein the mask aperture further comprises an adjustable flow restrictor for restricting the flow of gas from the nasal breathing cavity directly to ambient, wherein the adjustable flow restrictor is adjustable to maintain the positive end-expiratory pressure (PEEP) in the nasal breathing cavity of between 0.2 kPa and 1 kPa during the administering of high flow oxygen therapy to the patient.

33. The nasal respiratory mask according to claim 32, wherein a flow rate through the flow restrictor of the mask is adjusted to be at least 2 litres per minute, preferably at least 5 litres per minute.

34. The nasal respiratory mask of claim 20, wherein the mask frame has a generally domed shape.

35. The nasal respiratory mask of claim 20, wherein the mask cushion comprises a thermoplastic elastomer and/or silicone.

36. The nasal respiratory mask of claim 20, wherein the mask cushion and at least a perimeter of the mask frame are integrally formed of the same material.

37. The nasal respiratory mask of claim 20, wherein at least a portion of the mask frame comprises a substantially less flexible material than the material of the mask cushion.

38. The nasal respiratory mask of claim 20, wherein the mask cushion is inflatable and deflatable.

39. The nasal respiratory mask of claim 20, wherein the hose attachment portion is substantially centrally located on a vertical centre line of the mask frame.

40. The nasal respiratory mask of claim 20, wherein the hose attachment portion is located towards a lower end of the mask frame, preferably so as to be adjacent a middle of a user's mouth when worn.

41. The nasal respiratory mask of claim 20, comprising two of the mask apertures spaced substantially symmetrically about a vertical centre line of the mask frame.

42. The nasal respiratory mask of claim 41, wherein the two mask apertures are located towards a lower end of the mask frame so as to be adjacent either side of a user's mouth when worn.

43. The nasal respiratory mask of claim 20, wherein the mask frame is at least partially formed from a water permeable material.

44. The nasal respiratory mask of claim 43, wherein at least 50% of the mask frame is formed from the water permeable material.

45. The nasal respiratory mask of claim 43, wherein the water permeable material is permeable to liquid water and/or water vapour.

46. The nasal respiratory mask of claim 20, wherein the hose attachment portion comprises a swivel connector configured to provide relative rotation between the mask frame and the hose.

47. The nasal respiratory mask of claim 20, comprising a pair of opposing straps and/or harness extending from the mask frame.

48. The nasal respiratory mask of claim 20, further comprising a carbon dioxide monitoring line connector on the mask frame for attaching a carbon dioxide monitoring line and/or a carbon dioxide sensor.

49. The nasal respiratory mask of claim 48, further comprising a carbon dioxide sensor on the mask frame or further comprising a carbon dioxide monitoring line attached to the carbon dioxide monitoring line connector and a carbon dioxide sensor attached to the carbon dioxide monitoring line, optionally wherein the carbon dioxide monitoring line comprises a water permeable material.

50. The nasal respiratory mask of claim 20, further comprising a filter membrane arranged to cover the mask aperture, optionally wherein the filter membrane is arranged to cover at least half of the mask frame, and optionally wherein the filter membrane is arranged to cover a patient's mouth.

51. A nasal respiratory mask system comprising the nasal respiratory mask of claim 20 and a hose for attaching to the hose attachment portion of the nasal respiratory mask for delivering a supply of oxygen enriched air to the user.

52. The nasal respiratory mask system of claim 51, wherein the hose comprises a water permeable material.

53. The nasal respiratory mask system of claim 51, wherein the water permeable material is permeable to liquid water and/or water vapour.

54. The nasal respiratory mask system of claim 51, wherein the hose is malleable and/or comprises a malleable member, such that the hose is configured to be deformable and retain a given shape when the hose is manipulated.

55. A high flow oxygen therapy apparatus comprising:
the nasal respiratory mask system of claim 51; and
an oxygen enriched air supply coupled via the hose to the respiratory mask and configured to supply oxygen enriched air to a user.

56. The high flow oxygen therapy apparatus of claim 55, wherein the oxygen enriched air supply is configured to deliver a flow rate of at least 5 litres per minute to the user, and preferably a flow rate of between 30 and 60 litres per minute.

57. The high flow oxygen therapy apparatus of claim 55, wherein the oxygen enriched air supply is configured to deliver a flow rate of less than 70 litres per minute to the user.
